(19) United States
(12) Patent Application Publication
(10) Pub. No.: US 2025/0135459 A1
(43) Pub. Date: May 1, 2025

(54) Tray Device

(71) Applicants: Barbara Smith, Tempe, AZ (US); Ethan Marschall, Queen Creek, AZ (US); Jarrett Eshima, Phoenix, AZ (US)

(72) Inventors: Barbara Smith, Tempe, AZ (US); Ethan Marschall, Queen Creek, AZ (US); Jarrett Eshima, Phoenix, AZ (US)

(73) Assignee: Arizona Board of Regents on behalf of Arizona State University, Scottsdale, AZ (US)

(21) Appl. No.: 18/926,759

(22) Filed: Oct. 25, 2024

Related U.S. Application Data
(60) Provisional application No. 63/593,326, filed on Oct. 26, 2023.

(51) Int. Cl. B01L 9/00

(52) U.S. Cl. CPC B01L 9/00; B01L 2300/08

(57) ABSTRACT

Aspects of the present invention relate to a tray device including a frame having a top surface, a bottom surface, and a thickness, wherein the top surface of the frame has a first recessed region having a sidewall around at least a portion of a perimeter, and a ledge extending laterally from the bottom of the sidewall of the first recessed region, wherein the sidewall defines at least a portion of a first shape, a second recessed region having a sidewall around at least a portion of a perimeter, and a ledge extending laterally from the bottom of the sidewall of the second recessed region, wherein the sidewall defines at least a portion of a second shape, and wherein the depth of the first recessed region is deeper into the thickness of the tray than the depth of the second recessed region.

### CROSS-REFERENCE TO RELATED APPLICATIONS

This application claims priority to U.S. Provisional Application No. 63/593,326 filed on Oct. 26, 2023, incorporated herein by reference in its entirety.

### REFERENCE TO A “SEQUENCE LISTING,” SUBMITTED AS AN XML FILE

The Sequence Listing written in the xml file titled: “206339-0102-00US_SequenceListing.xml”; created on Oct. 11, 2024, and 11,672 bytes in size, is hereby incorporated by reference.

### BACKGROUND OF THE INVENTION

Imaging labware receptacles (e.g. a biodome or culture flask) presents numerous limitations to the user performing the imaging. In many cases, there is an inability to lay a receptacle flat against the stage of a microscope. Further, in many cases there is not a reproducible way of placing the receptacle in the same location on the stage, or imaging the same location within the receptacle. Lastly, multiplexing of numerous receptacles simultaneously presents the aforementioned issues but compounded across numerous receptacles.

Thus, there is a need in the art to develop an imaging tray device and imaging system that allow efficient, reproducible and multiplexed imaging of labware receptacles. The present invention meets this need.

### SUMMARY OF THE INVENTION

Aspects of the present invention relate to a tray device including a frame having a top surface, a bottom surface, and a thickness, wherein the top surface of the frame has a first recessed region having a sidewall around at least a portion of a perimeter, and a ledge extending laterally from the bottom of the sidewall of the first recessed region, wherein the sidewall defines at least a portion of a first shape, a second recessed region having a sidewall around at least a portion of a perimeter, and a ledge extending laterally from the bottom of the sidewall of the second recessed region, wherein the sidewall defines at least a portion of a second shape, and wherein the depth of the first recessed region is deeper into the thickness of the tray than the depth of the second recessed region.

In some embodiments, the device has a third recessed region within the top surface of the frame, wherein the third recessed region has a sidewall around at least a portion of a perimeter and a ledge extending laterally from the bottom of the sidewall, and wherein the sidewall defines at least a portion of a third shape.

In some embodiments, the depth of the second recessed region is deeper into the thickness of the tray than the depth of the third recessed region. In some embodiments, the first shape at least partially resembles the shape of a biodome device. In some embodiments, the second shape at least partially resembles the shape of a culture flask. In some embodiments, the third shape at least partially resembles the shape of a culture flask.

In some embodiments, the sidewall of the first recessed region has a height ranging from about 0.1 mm to about 10 mm. In some embodiments, the sidewall of the second recessed region has a height ranging from about 0.1 mm to about 10 mm. In some embodiments, the sidewall of the third recessed region has a height ranging from about 0.1 mm to about 10 mm.

In some embodiments, the first recessed region at least partially overlaps with the second recessed region. In some embodiments, the perimeter of the first recessed region is less than the perimeter of the second recessed region. In some embodiments, the first recessed region and the second recessed region are encompassed entirely within the perimeter of the third recessed region.

In some embodiments, the device has an opening that passes through the first, second, and third recessed regions. In some embodiments, the frame is composed of an opaque and rigid material. In some embodiments, the device has one or more handles extending up vertically from the top surface of the frame.

In some embodiments, the device is used for imaging a biological sample within a biodome. In some embodiments, the biological sample consists of a bacterial cell. In some embodiments, the biological sample consists of a mammalian cell.

Aspects of the present invention relate to a method of using a device of claim 1 for fluorescent imaging of a biological sample to measure volatile organic compounds (VOCs). In some embodiments, the method includes a dynamic headspace sampling methodology and computational modeling.

### BRIEF DESCRIPTION OF THE DRAWINGS

The following detailed description of embodiments of the invention will be better understood when read in conjunction with the appended drawings. It should be understood, however, that the invention is not limited to the precise arrangements and instrumentalities of the embodiments shown in the drawings.

FIG. 1A depicts a perspective view of an exemplary imaging tray according to aspects of the present invention. FIG. 1B depicts a top down view of an exemplary imaging tray according to aspects of the present invention. FIG. 1C depicts a side view of an exemplary imaging tray according to aspects of the present invention.

FIG. 1D depicts various views and dimensions for an exemplary imaging tray according to aspects of the present invention. FIG. 1E depicts a top down view with dimensions of an exemplary imaging tray according to aspects of the present invention.

FIG. 1F shows a perspective view of an exemplary imaging tray retaining a biodome for imaging and mounted on a microscopy stage according to aspects of the present invention. FIG. 1G shows a side view of an exemplary imaging tray retaining a biodome for imaging and mounted on a microscopy stage according to aspects of the present invention.

FIG. 2 depicts an illustrative computer architecture for a computer for practicing the various embodiments of the invention.

FIG. 3A to FIG. 3N show the design and development of the biodome. FIG. 3A shows the borosilicate glass culture vessel, termed the Biodome, containing 5 mL RPMI 1640 media. T-25 gas permeable flask caps were allowed to rest over the inlet and outlet of the vessel while in an incubator. FIG. 3B shows the placement of the Biodome during VOC sampling, showing the glass vessel is fully submerged in beads at 37° C. Also shown is the interface between the Biodome, TDT adapter, and TDT. A sterile rubber stopper and PTFE tape are used to create an airtight seal at the interface between the TDT and the adapter. FIG. 3C shows a 3D rendering showing the major components of the gas flow system including the hydrocarbon trap, flow meter, sterile filter, tubing, Biodome, TDT adapter, and TDT sampling tube. The compressed gas cylinder is not shown. FIG. 3D shows a schematic showing relevant dimensions (in mm; OD) for the Biodome culture vessel. FIG. 3E shows a representative GC×GC chromatogram spanning 400-2200 seconds in the first dimension and 0.5-2 seconds in the second dimension, following 24 hours of sampling SK-OV-3 ovarian adenocarcinoma cells. FIG. 3F shows fluid modeling in ANSYSM that demonstrates laminar flow conditions are maintained at flow rates≤20 mL/min (TDT adapter insert included). FIG. 3G shows the gauge pressure, and FIG. 3H shows velocity changes due to the inflowing gas are negligible at roughly half the Biodome height, limiting turbulent interactions with the surface of the cell culture media. FIG. 3I shows the results for a live/dead assay images in the Biodome showing viability across 0.5-4 days. A T-75 flask at t=0 (24 hours post seeding) is shown for reference. FIG. 3J shows the results for an independent live-dead assays performed using NucBlue™ (Hoechst 33342) and NucGreen™ ReadyProbes™ at 24-hour intervals, with live percentages estimated from 10 independent regions of the T-75 or Biodome culture vessels. No statistically significant differences in cell viability are observed demonstrating equivalence as compared to standard culture vessels. FIG. 3K shows a custom imaging tray designed in SolidWorks® 2019 and 3D printed to allow imaging using a Leica DMi8. FIG. 3L, FIG. 3M, and FIG. 3N show that typical SK-OV-3 morphology was visualized using fluorescent imaging and a live actin tracking stain (green color), Hoechst 33342 (blue color; live stain), and NucGreen (red color; dead stain), and no differences are observed using different culture vessels and growth environments. All brightfield and fluorescent images were taken using cells at passage<10.

FIG. 4A through FIG. 4D shows the volatilome characterization using the in vitro Biodome sampling tool. FIG. 4A shows a bar chart showing the number of VOCs detected, categorized according to their proposed origin. Variably present indicates the VOCs were observed inconsistently in two or more experimental conditions. FIG. 4B shows a PCA plot showing distinct clusters when considering the first two principal components, using the 384 features comprising the filtered volatilome. The first day SK-OV-3 volatilome was seen to resemble the media control more closely, as compared to the other three days, suggesting a 24 “flushing” period prior to collection may help with the reproducibility of in vitro mammalian volatilomes. FIG. 4C shows representative boxplots, and FIG. 4D shows line plots showing three low-abundance VOCs (ID<4) reproducibly and exclusively detected in the SK-OV-3 ovarian adenocarcinoma in vitro volatilome. Abundance values were calculated by integrating the peak area using the unique mass and log₁₀ transformed. The adjusted p value was calculated using a two-tailed student's t-test and corrected using the Benjamini-Hochberg procedure [Benjamini, Y., Hochberg, Y., Journal of the Royal statistical society: series B (Methodological) 57, 289-300 (1995)].

FIG. 5A through FIG. 5E shows glycolysis-mediated isotopic labeling in the Biodome. FIG. 5A shows a diagram showing the workflow implemented for the stable isotope labeling of glycolysis-derived VOCs. Representative mass spectrums comparing (FIG. 5B) labeled and unlabeled, (FIG. 5C) ¹³C-labeled and media control, and (FIG. 5D) ¹³C-labeled and flow system control were selected using the peak with the highest signal-to-noise ratio across the four days of sampling. Despite observing 2-Decen-1-ol in non-cellular control volatilomes, many low intensity fragments remained uniquely observed in the ¹³C-labeled spectra. Breakout images showing m/z ranges between 107-114, 122-128, and 137-147 amu were generated in R using raw mass spectral data. The proposed ¹³C-labeled structure is indicated in red, while annotations correspond to the unlabeled (¹²C) fragments. Fragments without an annotation are possibly derived from secondary bond rearrangements. FIG. 5E shows a proposed metabolic pathway for ¹³C incorporation into 2-Decen-1-ol.

FIG. 6A through FIG. 6D shows knockdown of CPT2 supports endogenous VOC production. FIG. 6A shows the expression of CPT2 quantified by RT-qPCR at the start of VOC collection (time=0) using biologically independent replicates. FIG. 6B shows the expression of CPT2 was independently mapped across the four days of VOC sampling following siRNA-mediated knockdown in SK-OV-3 cells at equal passage number using biologically independent replicates. FIG. 6C shows four VOCs not found in controls showing decreased abundance is consistent with the transient inhibition of β-oxidation via CPT2 knockdown, across 4 days of measurement. FIG. 6D shows boxplots showing significant or trending toward significant decreases in endogenous VOC abundance following integration by unique mass and log₁₀ transformation. P-values were adjusted using the Benjamini-Hochberg procedure [Benjamini, Y., Hochberg, Y., Journal of the Royal statistical society: series B (Methodological) 57, 289-300 (1995)].

FIG. 7A through FIG. 7D show the application of the Biodome tool to characterize the E. coli volatilome across the log and stationary phases of growth. FIG. 7A shows a Venn diagram showing the number of unique and common VOCs detected in the bacterial and broth conditions. Pie charts show the breakdown of VOC functional groups specific to each group. FIG. 7B shows a PCA analysis using high confidence VOCs presented in Table 2 showing the E. coli volatilome is distinct from the broth control. FIG. 7C shows line plots for four high confidence VOCs showing log₁₀ transformed abundance across three days of sampling. Day one represents the cumulative volatilome between 0-24 hours of growth, day 2 between 24-48 hours, and the final day capturing 48-72 hours. FIG. 7D shows boxplots for the same four VOCs showing expression of these metabolites are significantly elevated in the E. coli experimental condition, after correcting for multiple hypothesis testing using the Benjamini-Hochberg p-value adjustment procedure [Benjamini, Y., Hochberg, Y., Journal of the Royal statistical society: series B (Methodological) 57, 289-300 (1995)].

### DETAILED DESCRIPTION

It is to be understood that the figures and descriptions of the present invention have been simplified to illustrate elements that are relevant for a clear understanding of the present invention, while eliminating, for the purpose of clarity many other elements found in related systems and methods. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, exemplary materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate.

The terms “patient,” “subject,” “individual,” and the like are used interchangeably herein, and refer to any animal amenable to the systems, devices, and methods described herein. The patient, subject or individual may be a mammal, and in some instances, a human.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Imaging Tray Device and Imaging System

Aspects of the present invention relate to an imaging tray device for holding and positioning different types of receptacles including culture flasks, dishes, biodomes and other receptacles.

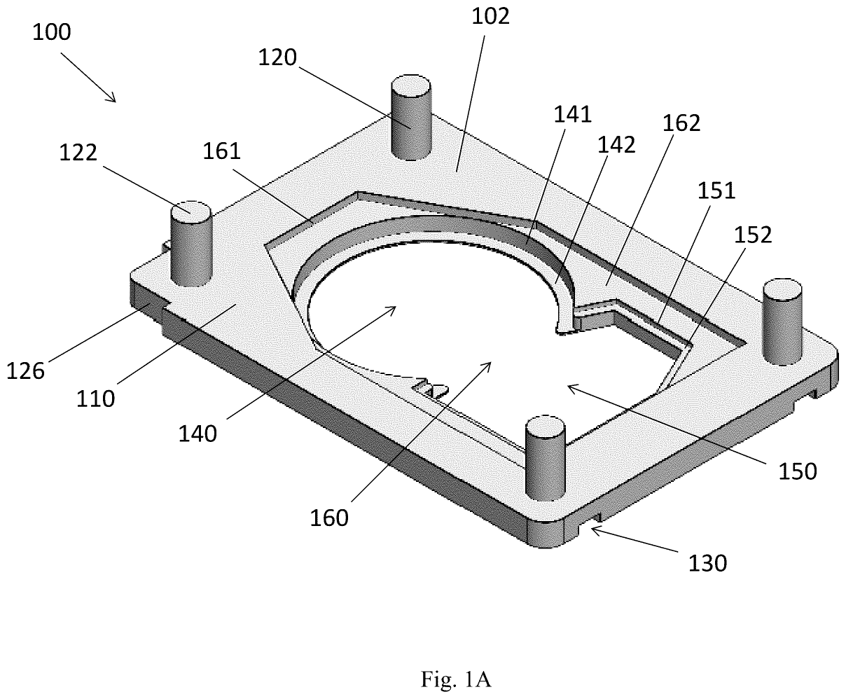

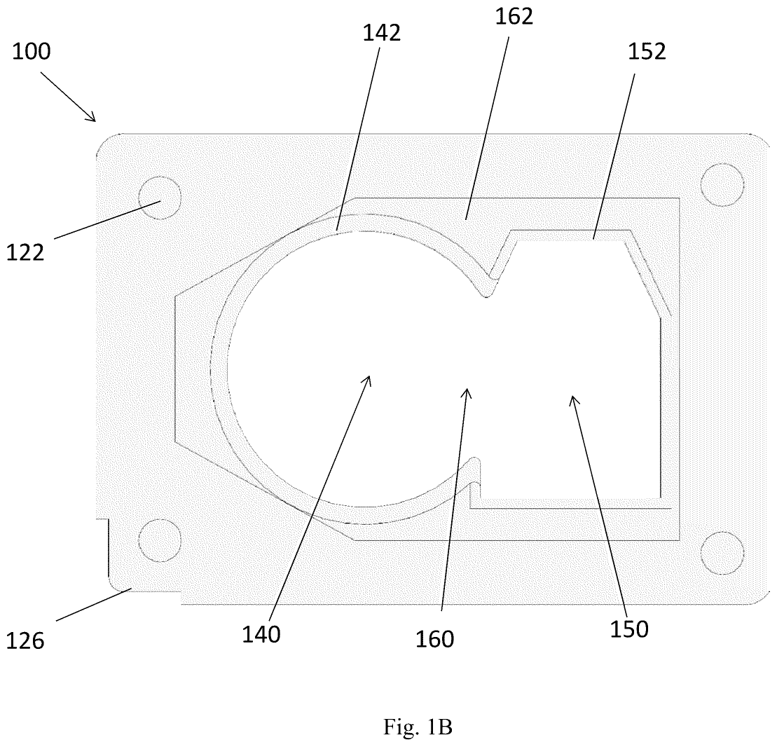

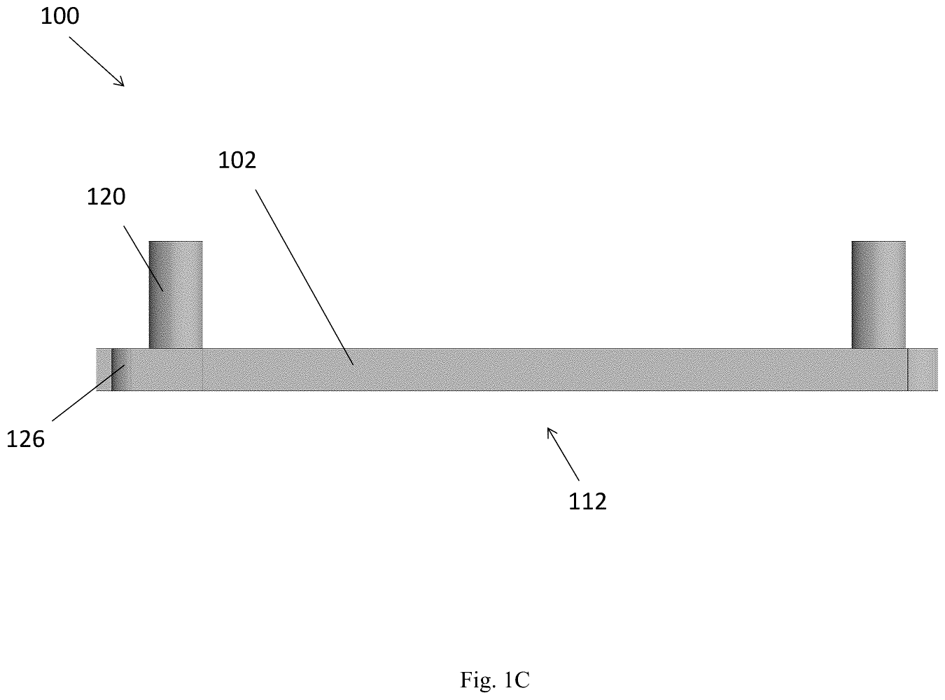

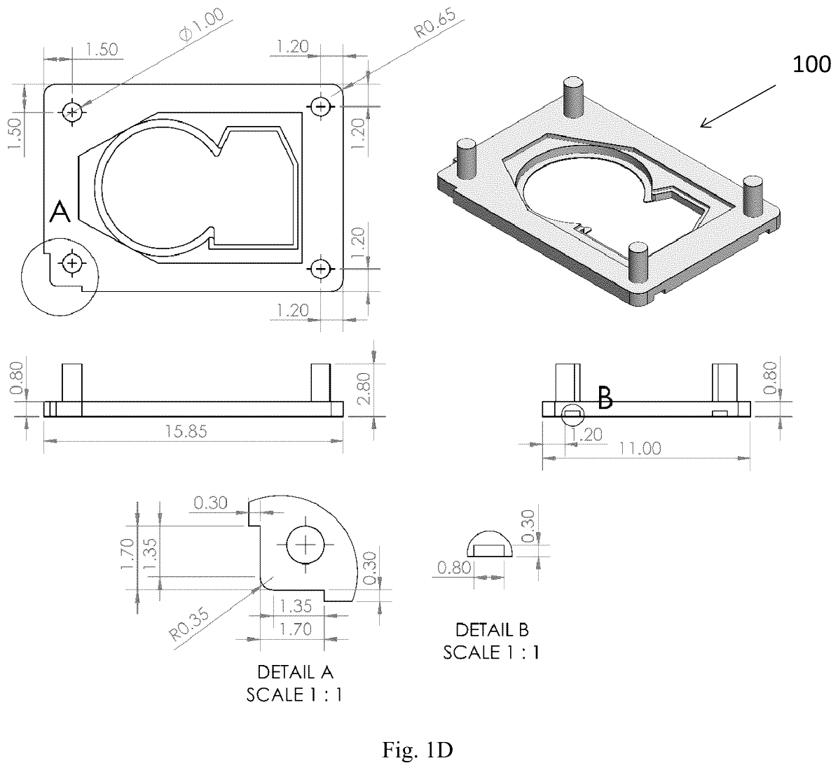

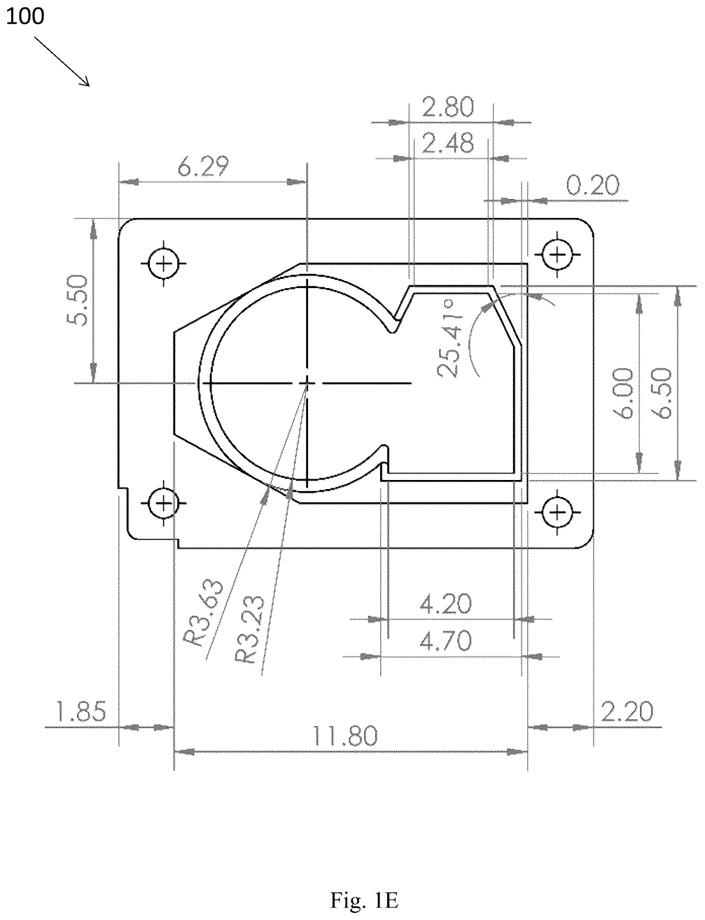

Referring now to FIGS. 1A-1C, shown is a perspective view of an exemplary imaging tray device 100 according to aspects of the present invention. In some embodiments, device 100 comprises a frame 102 having a top surface 110 and a bottom surface 112 with a thickness, and one or more recessed regions in the frame. In some embodiments, device 100 further comprises a plurality of handles 120 extending upward from top surface 110 of frame 102. In some embodiments, device 100 further comprises one or more cutout regions 126 in the frame and/or one or more indents 130 on the bottom surface of the frame.

In some embodiments, the one or more recessed regions comprise at least a first recessed region 140 having a sidewall 141 around at least a portion of a perimeter, and a ledge 142 extending laterally from the bottom of sidewall 142. In some embodiments, the one or more recessed regions comprises a second recessed region 150 having a sidewall 151 around at least a portion of a perimeter, and a ledge 152 extending laterally from the bottom of sidewall 152. In some embodiments, the one or more recessed regions comprises a third recessed region 160 having a sidewall 161 around at least a portion of a perimeter, and a ledge 162 extending laterally from the bottom of sidewall 162. It should be appreciated that the one or more recessed regions may comprise any number of recessed regions, configured in any arrangement on top surface 110 of device 100.

Each ledge of each recessed region forms a surface whereon a receptacle (e.g., a culture flask or biodome as discussed herein) may be fixedly and removably positioned or secured for imaging. In some embodiments, the device may then be placed on or affixed to an imaging platform (e.g., the tray or stage of a microscope). Each recessed region comprises a sidewall having a height ranging between 0.1 mm and 20 mm. In some embodiments, the height of sidewall 141, sidewall 151, or sidewall 161 are equal, or different. For example, in some embodiments, the height of sidewall 141 is equivalent to the height of sidewall 151, and greater than the height of sidewall 161. It should be appreciated that the sidewalls may be sized and shaped to accept any given receptacle known by one of ordinary level of skill in the art.

Each recessed region has sidewalls formed at least partially in the shape of, and sized for holding a receptacle (e.g., a labware, a flask, a Petri dish, a biodome). In some embodiments, the shape resembles the perimeter or outline formed by portions of the given receptable (e.g., a perimeter formed by the sidewalls and/or lower surface of a biodome device or flask). In some embodiments, the one or more recessed regions comprise sidewalls in a shape selected from any of circular, square, rectangular, trapezoidal, polygonal, pentagonal, hexagonal, cruciform, or any combination thereof. In some embodiments, first recessed region 140 comprises a round shape, and second recessed region 150 and third recessed region 160 comprise a polygonal or hexagonal shape. In some embodiments, first recessed region 140 is configured to receive and retain a biodome, second recessed region 150 is configured to receive and retain a T25 flask, and third recessed region 160 is configured to receive and retain a T75 flask. Although exemplary culture flask sizes are described, it should be appreciated that the recessed regions may be at least partially in the shape of and/or sized for holding any sized culture flask.

Any of the recessed regions may comprise smaller shapes, and sidewall heights, relative to one of the other recessed regions. Further, any of the recessed regions may be sized and shaped to fit at least partially within one of the other recessed regions. For example, first recessed region 140 and/or second recessed region 150 may have smaller shapes more with sidewalls more deeply recessed in top surface 110 of frame 102, whereas third recessed region 160 is a larger shape encompassing both first recessed region 140 and second recessed region 150, and has sidewalls less deeply recessed in top surface 110 of frame 102. Although described in FIG. 1A as first recessed region 140 and second recessed region 150 disposed within third recessed region 160, it should be appreciated that the one or more recessed regions may be positioned in any configuration on frame 102 including overlapping, partially overlapping, non-overlapping, adjacent, concentric, or any combination thereof.

Each recessed region may comprise one or more openings passing through tray 102. For example, in some embodiments, an opening passes through at least a portion first recessed region 140, and second recessed region 150.

Aspects of the present invention relate to a plurality of handles for device 100. Plurality of handles 120 extend upward from top surface 110 of tray 102 and are configure for grasping with a hand for moving the tray (e.g., on and off a microscope stage). In some embodiments, each handle of plurality of handles 120 comprises any of a a post, a cylindrical shape, a circular shape, a rectangular prism shape, a rectangular shape, a hexagonal shape, or any combination thereof. In some embodiments, each handle of plurality of handles 120 comprise a top surface 122.

In some embodiments, one or more cutout regions 126 comprise a cut-out region in the frame configured to engage one or more detent springs of a microscope or microscopy stage. In some embodiments, each indent of indents 130 comprises a cut-out region in the bottom surface of the frame configured to at least partially enclose one or more microscope or microscopy stage features. In some embodiments, device 100 further comprises markings on frame 102, top surface 110 or bottom surface 112 indicating any of: frame size, recessed region size, acceptable flask sizes, acceptable round lab-ware sizes, acceptable microscope and/or acceptable microscope stage, objective strength, or any combination thereof. In some embodiments, the markings are alphanumeric characters. In some embodiments the markings are embossed or debossed into top surface 110 and/or bottom surface 112. In some embodiments, the markings are any character or shape in any color.

Aspects of the present invention relate to dimensions for device 100. In some embodiments, frame 102 comprises a length ranging between 1 and 20 cm, a width ranging between 1 and 20 cm, and a height ranging between 1 mm and 5 cm. In some embodiments, each post of plurality of handles 120 comprises a height ranging between 1 mm and 5 cm, and a diameter or width ranging between 1 mm and 5 cm. In some embodiments, first recessed region 140, second recessed region 150 and/or third recessed region 160 comprise a diameter or width ranging between 1 cm and 10 cm. In some embodiments, first recessed region 140, second recessed region 150 and/or third recessed region 160 comprise a length ranging between 1 cm and 10 cm. In some embodiments, sidewall 141, sidewall 151, and/or sidewall 161 comprise a height ranging between 0.1 mm and 20 mm. In some embodiments, ledge 142, ledge 152, and/or ledge 152 comprises a width ranging between 0.1 mm and 10 mm. In some embodiments, each post of plurality of handles comprises a height ranging between 0.1 mm and 5 cm, and a diameter or width ranging between 0.1 mm and 5 cm.

Imaging tray device 100 may be comprised of, composed of, or manufactured from a variety of materials. For example, in some embodiments, device 100 comprises one or more materials selected from the group consisting of: ABS, PLA, TPU, PEEK, plastic, metal, acrylic, non-conductive materials, translucent materials, opaque materials, transparent materials, or combinations thereof. In some embodiments, device 100 is 3D printed, or plastic injection molded. In some embodiments, device 100 comprises one or more coatings selected from the group consisting of: anti-reflective coating, anti-microbial coating, waterproof coating, anti-radiation coating, reflective coating, mirror coating, or any combinations thereof.

Aspects of the present invention relate to an imaging system comprising the imaging tray device of the present invention. Referring now to FIG. 1F & FIG. 1G, shown is an exemplary imaging system 1000 comprising: imaging tray device 100, a biodome 200 comprising at least one inlet and at least one outlet, a pump, a microscope 400, and a computer (e.g. computer 500 described herein). In some embodiments, pump and/or microscope 400 are communicatively and electronically connected to computer 500. In some embodiments, a sample is positioned within biodome 200, and device 100 is positioned on microscope 400 (e.g. on the stage of the microscope). In some embodiments, biodome 200 is fixedly and removably positioned on first recessed surface 142 of recessed region 140. In some embodiments, pump 300 is fluidly connected to the one or more inlets of biodome 200 with one or more tubes. In some embodiments, microscope 400 images biodome 200 while fluid is being pumped through biodome 200 by pump 300. In some embodiments, one or more collectors is fluidly connected to the one or more outlets. In some embodiments, the one or more collectors comprise a VOC detection system, a mass spectrometer, a gas chromatography system, or combinations thereof. Imaging tray device 100 may be configured to position or affix any number of tools or sensors (e.g., sensors 565 discussed herein) to be used with the disclosed device and system or methods thereof. For example, one or more positioning arms removably or fixedly attached to frame 102 or plurality of handles 120 for positioning one or more tools or sensors.

Imaging system 1000 and/or imaging tray device 100 may be used with any device (e.g., a biodome), system or method described in U.S. Publication No. US20220331803A1, published on Oct. 20, 2022, the contents of which are incorporated herein by reference in their entirety. An exemplary biodome device is disclosed in U.S. Publication No. US20220331803A1. The biodome is a collection device for non-destructively gathering volatile organic compounds (VOCs) from biological samples. The device consists of a housing with an interior chamber that is configured to allow fluid to pass through it in a laminar flow, ensuring smooth and consistent flow and measurements. The housing has an upper and lower surface connected by a circular sidewall, creating a cylindrical volume in which a biological sample is placed. The device has inlets and outlets that allow for the introduction and removal of gases or fluids, arranged to maintain the laminar flow through the interior chamber. The interior chamber has features and dimensions that facilitate laminar flow, with a diameter of about 6.8 cm and a height of about 1.3 cm, but other sizes are possible to meet experimental needs. The biodome is made from materials that are capable of withstanding autoclaving and are heat-treated to reduce background VOC release. At least part of the housing is made from a transparent material, such as borosilicate glass, which allows for light transmission and imaging of the biological sample. The device is designed for reproducibility across multiple experiments and labs, making it suitable for collecting VOCs for applications such as disease biomarker detection.

The practice of the embodiments provided herein will employ, unless otherwise indicated, conventional techniques of molecular biology, microbiology, and immunology, which are within the skill of those working in the art. Such techniques are explained fully in the literature. Examples of particularly suitable texts for consultation include the following: Sambrook et al., Molecular Cloning: A Laboratory Manual, Third Ed., Cold Spring Harbor Laboratory, New York (2001); Ausubel et al., Current Protocols in Molecular Biology, John Wiley and Sons, Baltimore, MD (1999); Glover, ed., DNA Cloning, Volumes I and II (1985); Freshney, ed., Animal Cell Culture: Immobilized Cells and Enzymes (IRL Press, 1986); Kallen et al, Plant Molecular Biology-A Laboratory Manual (Ed. by Melody S. Clark; Springer-Verlag, 1997); Immunochemical Methods in Cell and Molecular Biology (Academic Press, London); Scopes, Protein Purification: Principles and Practice (Springer Verlag, N.Y., 2d ed. 1987); National Research Council (US) Committee on Methods of Producing Monoclonal Antibodies. Monoclonal Antibody Production. Washington (DC): National Academies Press (US); 1999; and Clausen H, et al. Glycosylation Engineering. 2017. In: Varki A, Cummings R D, Esko J D, et al., editors. Essentials of Glycobiology. 3rd edition. Cold Spring Harbor (NY): Cold Spring Harbor Laboratory Press; 2015-2017; and National Research Council (US) Committee on Revealing Chemistry through Advanced Chemical Imaging. Visualizing Chemistry: The Progress and Promise of Advanced Chemical Imaging. Washington (DC): National Academies Press (US); 2006. 3, Imaging Techniques: State of the Art and Future Potential.

### Computing Device

In some aspects of the present invention, software executing the instructions provided herein may be stored on a non-transitory computer-readable medium, wherein the software performs some or all of the steps of the present invention when executed on a processor.

Aspects of the invention relate to algorithms executed in computer software. Though certain embodiments may be described as written in particular programming languages, or executed on particular operating systems or computing platforms, it is understood that the system and method of the present invention is not limited to any particular computing language, platform, or combination thereof. Software executing the algorithms described herein may be written in any programming language known in the art, compiled, or interpreted, including but not limited to C, C++, C#, Objective-C, Java, JavaScript, MATLAB, Python, PHP, Perl, Ruby, or Visual Basic. It is further understood that elements of the present invention may be executed on any acceptable computing platform, including but not limited to a server, a cloud instance, a workstation, a thin client, a mobile device, an embedded microcontroller, a television, or any other suitable computing device known in the art.

Parts of this invention are described as software running on a computing device. Though software described herein may be disclosed as operating on one particular computing device (e.g. a dedicated server or a workstation), it is understood in the art that software is intrinsically portable and that most software running on a dedicated server may also be run, for the purposes of the present invention, on any of a wide range of devices including desktop or mobile devices, laptops, tablets, smartphones, watches, wearable electronics or other wireless digital/cellular phones, televisions, cloud instances, embedded microcontrollers, thin client devices, or any other suitable computing device known in the art.

Similarly, parts of this invention are described as communicating over a variety of wireless or wired computer networks. For the purposes of this invention, the words “network”, “networked”, and “networking” are understood to encompass wired Ethernet, fiber optic connections, wireless connections including any of the various 802.11 standards, cellular WAN infrastructures such as 3G, 4G/LTE, or 5G networks, Bluetooth®, Bluetooth® Low Energy (BLE) or Zigbee® communication links, or any other method by which one electronic device is capable of communicating with another. In some embodiments, elements of the networked portion of the invention may be implemented over a Virtual Private Network (VPN).

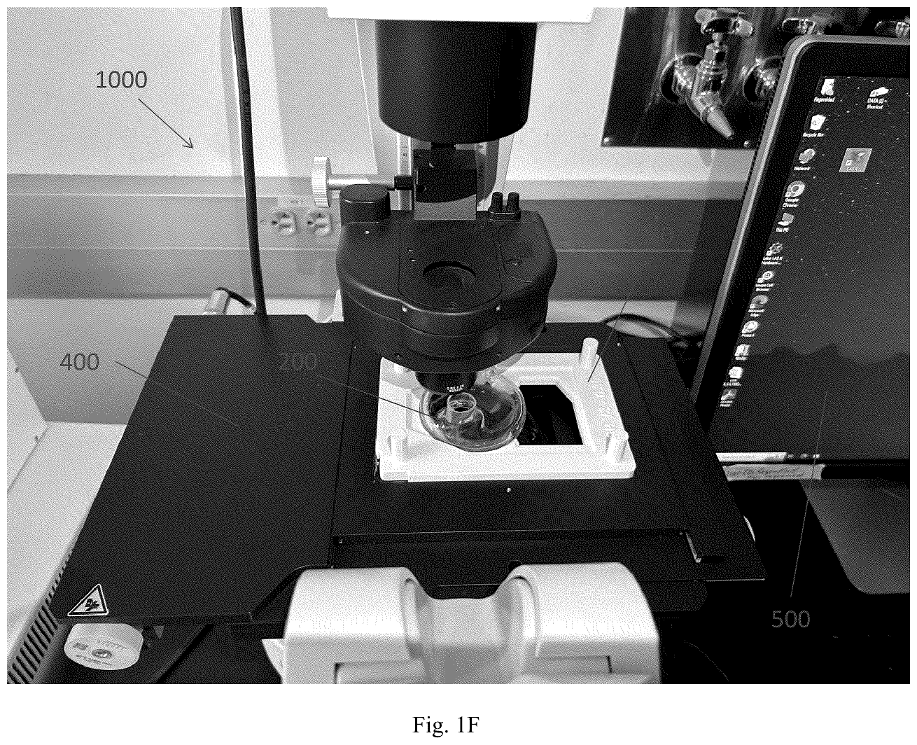

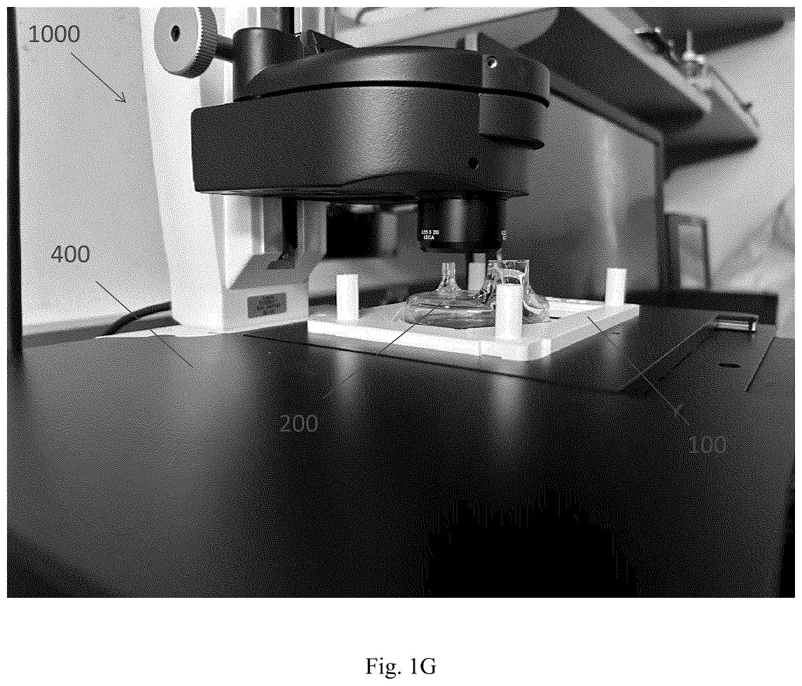

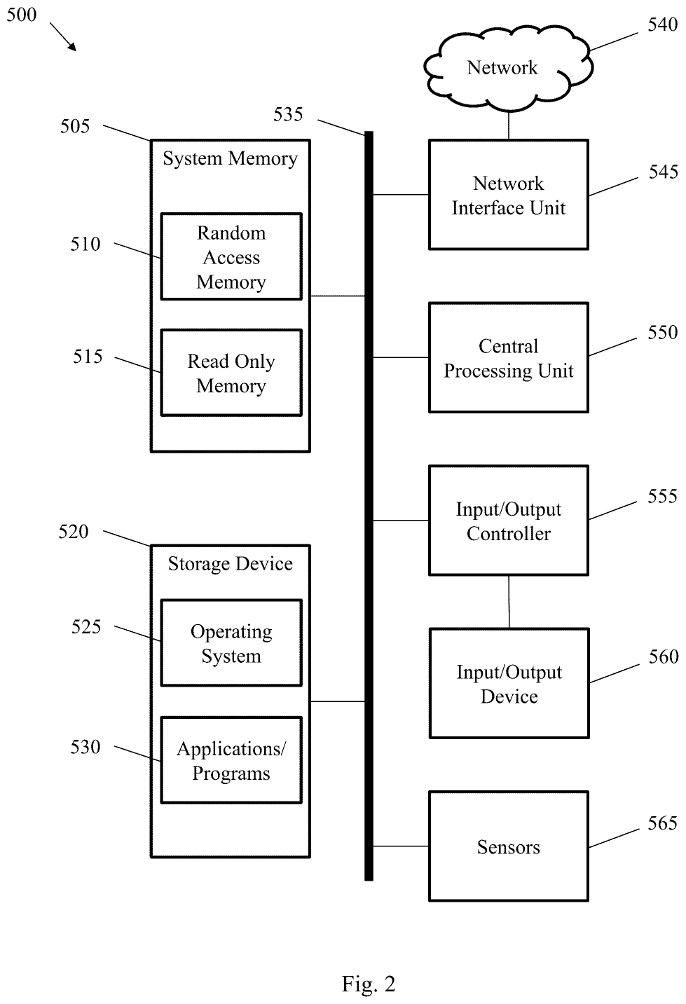

FIG. 2 and the following discussion are intended to provide a brief, general description of a suitable computing environment in which the invention may be implemented. While the invention is described above in the general context of program modules that execute in conjunction with an application program that runs on an operating system on a computer, those skilled in the art will recognize that the invention may also be implemented in combination with other program modules.

Generally, program modules include routines, programs, components, data structures, and other types of structures that perform particular tasks or implement particular abstract data types. Moreover, those skilled in the art will appreciate that the invention may be practiced with other computer system configurations, including hand-held devices, multiprocessor systems, microprocessor-based or programmable consumer electronics, minicomputers, mainframe computers, and the like. The invention may also be practiced in distributed computing environments where tasks are performed by remote processing devices that are linked through a communications network. In a distributed computing environment, program modules may be located in both local and remote memory storage devices.

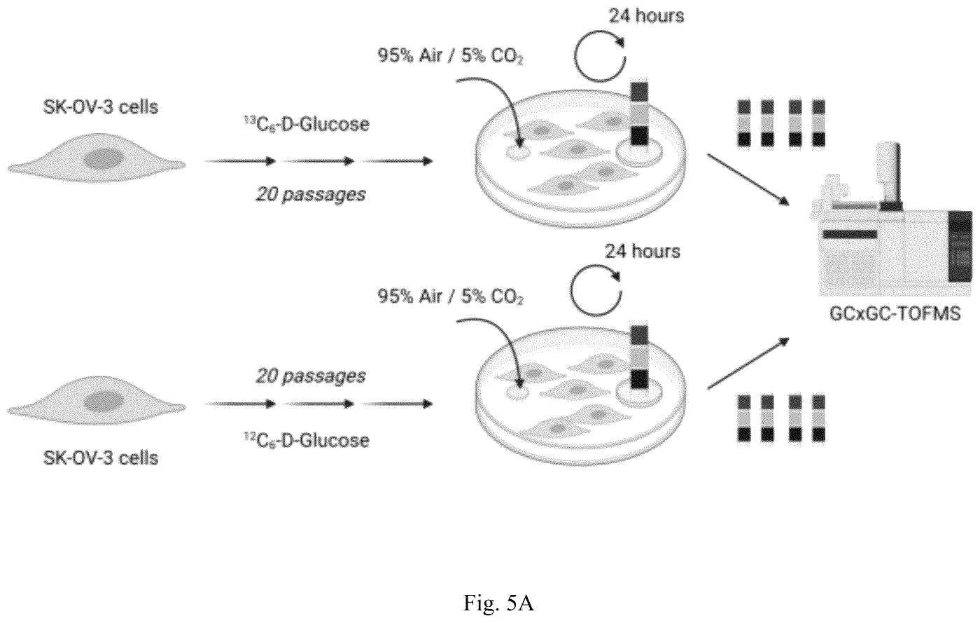

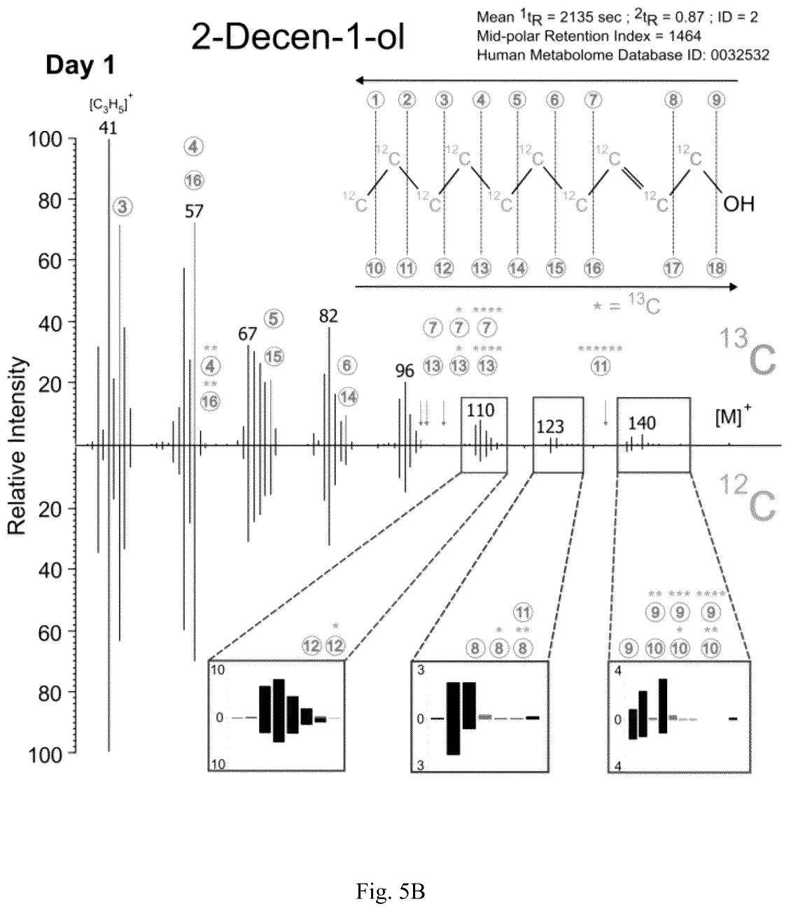

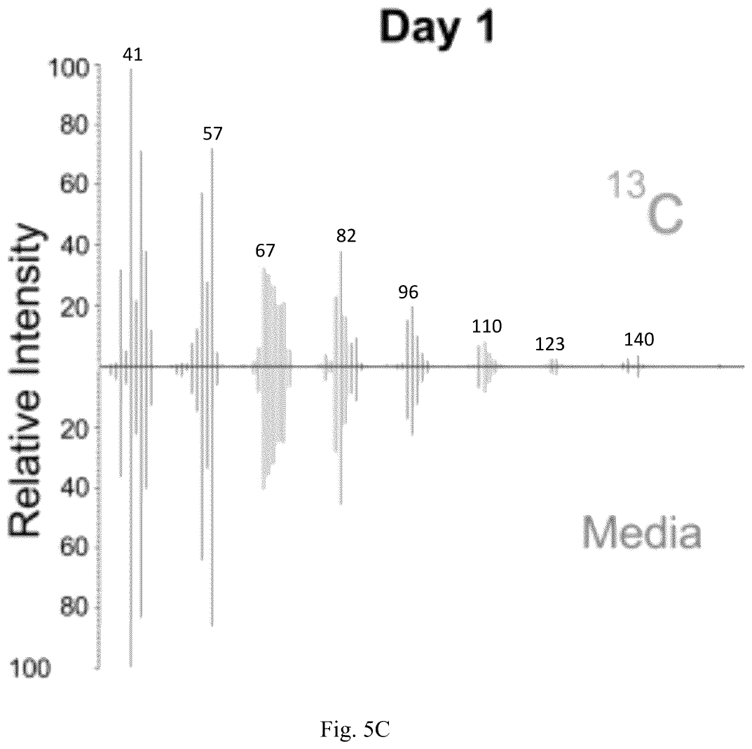

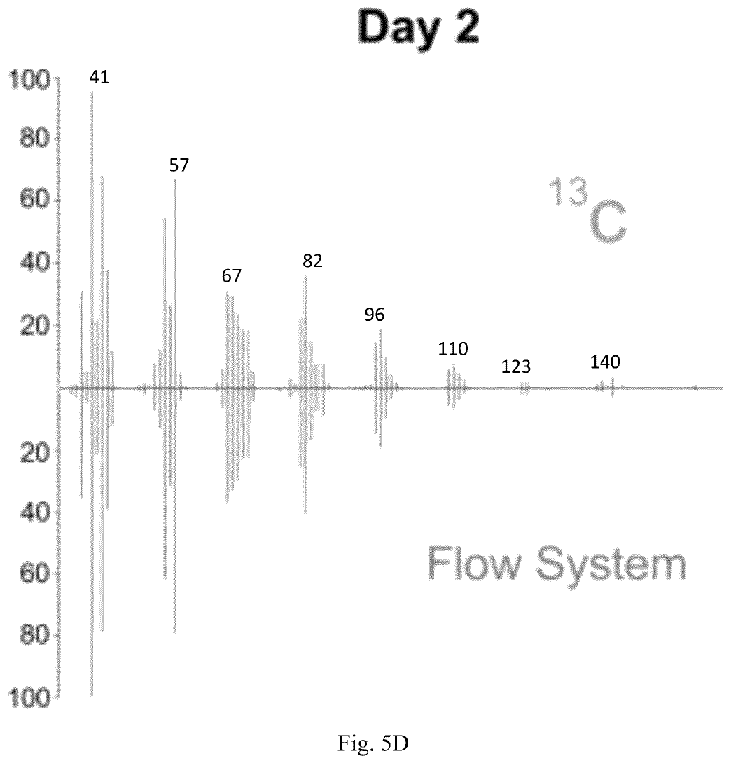

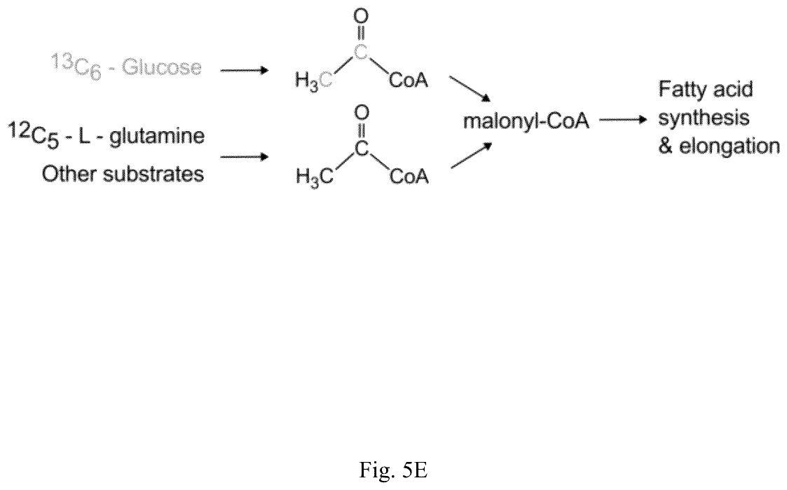

FIG. 2 depicts an illustrative computer architecture for a computer 500 for practicing the various embodiments of the invention. The computer architecture shown in FIG. 5 illustrates a conventional personal computer, including a central processing unit 550 (“CPU”), a system memory 505, including a random access memory 510 (“RAM”) and a read-only memory (“ROM”) 515, and a system bus 535 that couples the system memory 505 to the CPU 550. A basic input/output system containing the basic routines that help to transfer information between elements within the computer, such as during startup, is stored in the ROM 515. The computer 500 further includes a storage device 520 for storing an operating system 525, application/program 530, and data.

The storage device 520 is connected to the CPU 550 through a storage controller (not shown) connected to the bus 535. The storage device 520 and its associated computer-readable media provide non-volatile storage for the computer 500. Although the description of computer-readable media contained herein refers to a storage device, such as a hard disk or CD-ROM drive, it should be appreciated by those skilled in the art that computer-readable media can be any available media that can be accessed by the computer 500.

By way of example, and not to be limiting, computer-readable media may comprise computer storage media. Computer storage media includes volatile and non-volatile, removable and non-removable media implemented in any method or technology for storage of information such as computer-readable instructions, data structures, program modules or other data. Computer storage media includes, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the computer.

According to various embodiments of the invention, the computer 500 may operate in a networked environment using logical connections to remote computers through a network 540, such as TCP/IP network such as the Internet or an intranet. The computer 500 may connect to the network 540 through a network interface unit 545 connected to the bus 535. It should be appreciated that the network interface unit 545 may also be utilized to connect to other types of networks and remote computer systems.

The computer 500 may also include an input/output controller 555 for receiving and processing input from a number of input/output devices 560, including a keyboard, a mouse, a touchscreen, a camera, a microphone, a controller, a joystick, or other type of input device. Similarly, the input/output controller 555 may provide output to a display screen, a printer, a speaker, or other type of output device. The computer 500 can connect to the input/output device 560 via a wired connection including, but not limited to, fiber optic, Ethernet, or copper wire or wireless means including, but not limited to, Wi-Fi, Bluetooth, Near-Field Communication (NFC), infrared, or other suitable wired or wireless connections.

As mentioned briefly above, a number of program modules and data files may be stored in the storage device 520 and/or RAM 510 of the computer 500, including an operating system 525 suitable for controlling the operation of a networked computer. The storage device 520 and RAM 510 may also store one or more applications/programs 530. In particular, the storage device 520 and RAM 510 may store an application/program 530 for providing a variety of functionalities to a user. For instance, the application/program 530 may comprise many types of programs such as a word processing application, a spreadsheet application, a desktop publishing application, a database application, a gaming application, internet browsing application, electronic mail application, messaging application, and the like. According to an embodiment of the present invention, the application/program 530 comprises a multiple functionality software application for providing word processing functionality, slide presentation functionality, spreadsheet functionality, database functionality and the like.

The computer 500 in some embodiments can include a variety of sensors 565 for monitoring the environment surrounding and the environment internal to the computer 500. These sensors 565 can include a Global Positioning System (GPS) sensor, a photosensitive sensor, a gyroscope, a magnetometer, thermometer, a proximity sensor, an accelerometer, a microphone, biometric sensor, barometer, humidity sensor, radiation sensor, or any other suitable sensor.

### NUMERATED EMBODIMENTS

Embodiment 1: A tray device, comprising a frame comprising a top surface, a bottom surface, and a thickness,
wherein the top surface of the frame comprises:
   a first recessed region having a sidewall around at least a portion of a perimeter, and a ledge extending laterally from the bottom of the sidewall of the first recessed region, wherein the sidewall defines at least a portion of a first shape;
   a second recessed region having a sidewall around at least a portion of a perimeter, and a ledge extending laterally from the bottom of the sidewall of the second recessed region, wherein the sidewall defines at least a portion of a second shape; and
wherein the depth of the first recessed region is deeper into the thickness of the tray than the depth of the second recessed region.

Embodiment 2: The device of any previous embodiment, further comprising a third recessed region within the top surface of the frame,
wherein the third recessed region has a sidewall around at least a portion of a perimeter and a ledge extending laterally from the bottom of the sidewall, and
wherein the sidewall defines at least a portion of a third shape.

Embodiment 3: The device of any previous embodiment, wherein the depth of the second recessed region is deeper into the thickness of the tray than the depth of the third recessed region.

Embodiment 4: The device of any previous embodiment, wherein the first shape at least partially resembles the shape of a biodome device.

Embodiment 5: The device of any previous embodiment, wherein the second shape at least partially resembles the shape of a culture flask.

Embodiment 6: The device of any previous embodiment, wherein the third shape at least partially resembles the shape of a culture flask.

Embodiment 7: The device of any previous embodiment, wherein the sidewall of the first recessed region has a height ranging from about 0.1 mm to about 10 mm.

Embodiment 8: The device of any previous embodiment, wherein the sidewall of the second recessed region has a height ranging from about 0.1 mm to about 10 mm.

Embodiment 9: The device of any previous embodiment, wherein the sidewall of the third recessed region has a height ranging from about 0.1 mm to about 10 mm.

Embodiment 10: The device of any previous embodiment, wherein the first recessed region at least partially overlaps with the second recessed region.

Embodiment 11: The device of any previous embodiment, wherein the perimeter of the first recessed region is less than the perimeter of the second recessed region.

Embodiment 12: The device of any previous embodiment, wherein the first recessed region and the second recessed region are encompassed entirely within the perimeter of the third recessed region.

Embodiment 13: The device of any previous embodiment, further comprising an opening that passes through the first, second, and third recessed regions.

Embodiment 14: The device of any previous embodiment, wherein the frame comprises an opaque and rigid material.

Embodiment 15: The device any previous embodiment, further comprising one or more handles extending up vertically from the top surface of the frame.

Embodiment 16: The device of any previous embodiment, wherein it is used for imaging a biological sample within a biodome.

Embodiment 17: The device of any previous embodiment, wherein the imaging is fluorescent imaging.

Embodiment 18: The device of any previous embodiment, wherein the biological sample consists of a bacterial cell.

Embodiment 19: The device of any previous embodiment, wherein the bacterial cell is selected from Escherichia coli (E. coli), Bacillus subtilis, Pseudomonas aeruginosa, Mycobacterium tuberculosis, Streptomyces coelicolor, Salmonella enterica, Streptococcus species, Streptococcus pyogenes, Streptococcus pneumoniae, Lactococcus lactis, Mycobacterium tuberculosis, Clostridium species, Staphylococcus aureus, Rhodobacter species, Agrobacterium tumefaciens, vibrio species, Vibrio cholerae, Mycobacterium smegmatis, Caulobacter crescentus, Bacteroides thetaiotaomicron, Listeria monocytogenes, Shewanella oneidensis, Bacillus thuringiensis, Mycoplasma pneumoniae, Campylobacter jejuni, Geobacter sulfurreducens, Enterococcus faecalis, Actinobacteria, Enterococcus, Actinomyces, Lactobacillales, Actinomyces israelii, Listeria, Bacillales, Nocardia, Bacillus, Nocardia asteroids, Clostridium, Nocardia brasiliensis, Clostridium acetobutylicum, Propionibacterium acnes, Clostridium aerotolerans, Rhodococcus equi, Clostridium argentinense, Sarcina, Clostridium baratii, Solobacterium moorei, Clostridium beijerinckii, Staphylococcus, Clostridium bifermentans, Staphylococcus aureus, Clostridium botulinum, Staphylococcus capitis, Clostridium butyricum, Staphylococcus caprae, Clostridium cadaveris, Staphylococcus epidermidis, Clostridium cellulolyticum, Staphylococcus haemolyticus, Clostridium chauvoei, Staphylococcus hominis, Clostridium clostridioforme, Staphylococcus lugdunensis, Clostridium colicanis, Staphylococcus Musca,e Clostridium difficile, Staphylococcus nepalensis, Clostridium estertheticum, Staphylococcus pettenkoferi, Clostridium fallax, Staphylococcus saprophyticus, Clostridium formicaceticum, Staphylococcus succinus, Clostridium histolyticum, Staphylococcus warneri, Clostridium innocuum, Staphylococcus xylosus, Clostridium kluyveri, Strangles, Clostridium ljungdahlii, Streptococcus, Clostridium novyi, Streptococcus agalactiae, Clostridium paraputrificum, Streptococcus anginosus, Clostridium perfringens, Streptococcus bovis, Clostridium phytofermentans, Streptococcus canis, Clostridium piliforme, Streptococcus iniae, Clostridium ragsdalei, Streptococcus lactarius, Clostridium ramosum, Streptococcus mitis, Clostridium septicum, Streptococcus mutans, Clostridium sordellii, Streptococcus oralis, Clostridium sporogenes, Streptococcus parasanguinis, Clostridium sticklandii, Streptococcus peroris, Clostridium tertium, Streptococcus pneumoniae, Clostridium tetani, Streptococcus pyogenes, Clostridium thermosaccharolyticum, Streptococcus ratti, Clostridium tyrobutyricum, Streptococcus salivarius, Corynebacterium, Streptococcus sanguinis, Corynebacterium bovis, Streptococcus sobrinus, Corynebacterium diphtheriae, Streptococcus suis, Corynebacterium granulosum, Streptococcus salivarius thermophilus, Corynebacterium jeikeium, Streptococcus uberis, Corynebacterium minutissimum, Streptococcus vestibularis, Corynebacterium renale, Streptococcus viridans, Acetic acid bacteria, Fusobacterium necrophorum, Acinetobacter baumannii, Fusobacterium nucleatum, Agrobacterium tumefaciens, Fusobacterium polymorphum, Anaerobiospirillum, Haemophilus haemolyticus, Bacteroides, Haemophilus influenzae, Bacteroides fragilis, Helicobacter, Bdellovibrio, Helicobacter pylori, Brachyspira, Klebsiella pneumoniae, Cardiobacterium hominis, Legionella, Coxiella burnetii, Legionella pneumophila, Cyanobacteria, Leptotrichia buccalis, Cytophaga, Megamonas, Dialister, Megasphaera, Enterobacter, Moraxella, Enterobacter cloacae, Moraxella bovis, Enterobacteriaceae, Moraxella catarrhalis, Escherichia, Moraxella osloensis, Escherichia coli, Morganella morganii, Pseudomonas geniculate, Negativicutes, Rickettsia rickettsii, Neisseria gonorrhoeae, Salmonella, Neisseria meningitidis, Salmonella enterica, Neisseria sicca, Pectinatus, Selenomonadales, Propionispora, Serratia marcescens, Proteobacteria, Shigella, Proteus mirabilis, Spirochaetes, Proteus penneri, Spirochaetaceae, Pseudomonas, Sporomusa, Pseudomonas aeruginosa, Steno trophomonas, Streptococcus gordonii, Vampirococcus Verminephrobacter, Vibrio cholerae, Wolbachia, and Zymophilus.

Embodiment 20: The device of any previous embodiment, wherein the biological sample consists of a mammalian cell.

Embodiment 21: The device of any previous embodiment, wherein the mammalian cell is selected from a SK-OV-3 ovarian adenocarcinoma cell, Chinese hamster ovary (CHO), mouse myeloma derived NS0 and Sp2/0 cells, human embryonic kidney cells (HEK293), and human embryonic retinoblast-derived PER.C6 cells, HeLa, CHO, NIH/3T3, A549, MCF-7, PC-12, Caco-2, Jurkat, SH-SY5Y, U-2 OS, THP-1, SK-N-SH, RAW 264.7, LNCaP, MDCK, HepG2, C2C12, Vero, Sf9, and COS-7.

Embodiment 22: A method of using a device from any previous embodiment for fluorescent imaging of a biological sample to measure volatile organic compounds (VOCs).

Embodiment 23: The method of embodiment 22, further comprising a dynamic headspace sampling methodology and computational modeling.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore are not to be construed as limiting in any way the remainder of the disclosure.

### Example 1: Device to Hold and Image Cell Culture Flasks and Biodome Technologies

The disclosed device was born out of limitations encountered when imaging the biodome (and other receptacles) using a commercial microscope. The disclosed device has been specifically designed to provide a method for imaging contents within “the biodome”, including mammalian and bacterial cells. Prior to the design and development of this platform, there was an inability to lay the biodome flat for imaging the contents within the biodome. The disclosed device provides a microscope stage replacement or accessory which features a cut-out for positioning a single or multiplexed biodome unit. In addition, in some embodiments, this tool has been designed and developed with inlets to account for imaging commercially available T25 and T75 cell culture flasks. Furthermore, the positioning of the cutouts within this accessory enables controlled positioning of the biodome or commercially available cell culture flasks to allow the scientist or clinician to repeatably locate specific regions of interest for imaging.

This disclosed device was created as a stage replacement or adapter to commercially available stages to allow imaging using a Leica DMi8 microscope. This accessory enables imaging of the biodome (potentially single or multiplexed) as well as commercially available T25 and T75 cell culture flasks, and other lab-wares and receptacles. In some embodiments, the disclosed device provides a microscope platform to enable consistent and high resolution images of the contents (e.g. cells or bacteria) within the biodome. Due to the current manufacturing process for the biodome (and many other lab-wares and receptacles using blown glass)—the glass thickness is slightly variable across the receptacle, creating a requirement and need for a stage that improves the image quality of the contents within the biodome or receptacle.

The disclosed device was designed in SolidWorks® 2019 and 3D printed to allow imaging using a Leica DMi8 microscope. The prototype of the disclosed device was 3D printed with PRUSA MK3S+ with hatchbox PLA filament.

### Example 2: An Engineered Culture Vessel and Flow System to Improve the In Vitro Analysis of Volatile Organic Compounds

Volatile organic compounds (VOCs) are an important subset of an organism's metabolome, yet in vitro techniques for the analysis of these small molecules vary substantially in practice, restricting the interpretation of study findings. Disclosed in this example is an engineered culture tool and system to enhance analyte sensitivity by integrating dynamic headspace sampling methodology and a validation of device functionality utilizing computational modeling and fluorescent imaging of mammalian cell culture. Seven VOCs not found in the media or exogenously derived from the sampling method (typical pitfalls with in vitro analysis) were identified. Endogenous VOC production was validated using: (i) glycolysis-mediated stable isotopic labeling techniques using ¹³C₆-D-glucose and (ii) RNA interference (RNAi) to selectively knockdown β-oxidation via silencing of CPT2. Isotope labeling reveals 2-Decen-1-ol as endogenously derived with glucose as a carbon source and, through RNAi, evidence supporting endogenous production of 2-ethyl-1-hexene, dodecyl acrylate, tridecanoic acid methyl ester and a low abundance alkene (C17) was found. To demonstrate broad applicability, VOC production was assessed during the log and stationary phases of growth in ampicillin-resistant DH5α Escherichia coli and identified six endogenous VOCs not previously reported. The findings emphasize the improved capabilities for in vitro volatile metabolomics and provide a platform for the standardization of methodology.

Volatile metabolites, part of the broad class of compounds known as volatile organic compounds (VOCs), are products of cellular metabolism with low molecular weight and a sufficient vapor pressure to exist as a gas at standard temperature and pressure. VOCs have long been recognized to confer information about biological processes in health and disease [Sulway et al., The Lancet 296, 736-740 (1970); Wang, Z. et al., Journal of breath research 7, 037109 (2013); Williams, H., et al., The Lancet 333, 734 (1989)], yet reliable methods for the study of these molecules in vitro have lagged behind. Works overcoming the challenges associated with in vitro sampling methodology have provided evidence for the extensive roles of volatile metabolites in biology, including but not limited to, serving as putative disease biomarkers [Sponring, A. et al., Anticancer research 29, 419-426 (2009); Abaffy, T. et al., Metabolomics 9, 998-1008 (2013); Davis, T. J. et al., Msphere 5, e00843-e00820 (2020)], extracellular signaling molecules in archaea, fungi, bacteria, protists, plants, and animals [Weisskopf, L. et al., Nature Reviews Microbiology 19, 391-404 (2021); Bitas, V., et al., Molecular Plant-Microbe Interactions 26, 835-843 (2013); Song, G. C. et al., Environmental Microbiology 21, 940-948 (2019); Ditengou, F. A. et al., Nature communications 6, 6279 (2015); Becher, P. G. et al., Nature Microbiology 5, 821-829 (2020); Ponnusamy, L. et al., Proceedings of the National Academy of Sciences 105, 9262-9267 (2008); Schulz-Bohm, K. et al., The ISME journal 11, 817-820 (2017); Baldwin, I. T. et al., Science 311, 812-815 (2006); Dudareva, N. et al., New Phytologist 198, 16-32 (2013); Traxler, S. et al., Scientific reports 8, 14857 (2018)], and drivers of inter- and intra-kingdom phenotype and function [Weisskopf, L. et al., Nature Reviews Microbiology 19, 391-404 (2021); Song, G. C. et al., Environmental Microbiology 21, 940-948 (2019); Ditengou, F. A. et al., Nature communications 6, 6279 (2015); Becher, P. G. et al., Nature Microbiology 5, 821-829 (2020); Ebadzadsahrai, G. et al., Frontiers in Microbiology 11, 1035 (2020); Farag, M. A. et al., Nature protocols 12, 1359-1377 (2017); Kesarwani, M. et al., PLoS pathogens 7, e1002192 (2011); Kim, K. S. et al., Nature communications 4, 1809 (2013)]. Despite the insights gained, nearly all approaches suffer from one or more of the following major limitations: i) detection of exogenous VOCs from plastics or the sampling environment [Farag, M. A. et al., Nature protocols 12, 1359-1377 (2017); Schallschmidt, K. et al., Journal of chromatography B 1006, 158-166 (2015); Jia, Z. et al., Metabolites 9, 52 (2019)], ii) low analyte sensitivity and poor signal-to-noise relative to VOCs originating from non-endogenous sources, as a consequence of indirect [Farag, M. A. et al., Nature protocols 12, 1359-1377 (2017); Groenhagen, U. et al., Beilstein Journal of Organic Chemistry 10, 1421-1432 (2014)] and passive (equilibrium-based) sampling techniques [Schallschmidt, K. et al., Journal of chromatography B 1006, 158-166 (2015); Thriumani, R. et al., BMC cancer 18, 1-7 (2018); Reese, K. L., Scientific reports 10, 1-7 (2020)] and/or iii) disrupt culture viability [Krall, L., Huege, J. et. al., Journal of Chromatography B 877, 2952-2960 (2009); Bolling, C. et al., Plant physiology 139, 1995-2005 (2005); Franchina, F. A. et al., Analytica chimica acta 1066, 146-153 (2019)], limiting interpretation in response to environmental stimuli or perturbation. Thus, in vitro VOC studies would largely benefit from a standardized method and tool to reduce signal from contaminants, enhance sensitivity, and maintain viability for time-dependent applications.

Previous groups have recognized these limitations and worked to develop a broad family of approaches known as purge-and-trap or dynamic headspace sampling (DHS) [Sponring, A. et al., Anticancer research 29, 419-426 (2009); Schulz, S. et al., Tetrahedron 60, 3863-3872 (2004); Blom, D. et al., Environmental microbiology 13, 3047-3058 (2011); Ahmed, W. M. et al., Analyst 143, 4155-4162 (2018); Franchina, F. A. et al., Journal of separation science 43, 1790-1799 (2020); Ochiai, N. et al., Journal of Chromatography A 1371, 65-73 (2014)]. These approaches involve the continuous sampling or removal of the headspace, often trapping the VOCs on sorbent material fixed to solid support [Ochiai, N. et al., Journal of Chromatography A 1371, 65-73 (2014); Oliver-Pozo, C., Journal of agricultural and food chemistry 67, 2086-2097 (2019); Ha, J. et al., Food chemistry 142, 79-86 (2014); Maurer, D. L. et al., Scientific reports 9, 12103 (2019)], or directly analyzing the extracted gas by coupling the sampling methodology to a mass spectrometer [Traxler, S. et al., Journal of Breath Research 12, 041001 (2018); Rosenthal, K. et al., Analytical Methods 13, 5441-5449 (2021); Bunge, M. et al., Applied and environmental microbiology 74, 2179-2186 (2008)] or sensor array [Thriumani, R. et al., BMC cancer 18, 1-7 (2018); Lavra, L. et al., Scientific reports 5, 1-2 (2015)]. DHS has been shown to yield a significant increase in signal when considering the volatilome emitted from fruit extract [Silva, R. C. et al., Chromatographia 60, 687-691 (2004); Dias, R. P. et al., Journal of Chromatography Open 3, 100075 (2023)] and other complex matrices [Stefanuto, P. H. et al., Journal of Chromatography A 1507, 45-52 (2017)]. Despite the advantages of and advances in DHS methodology, translation to an in vitro biological setting has been limited and a standardized approach does not yet exist [Sponring, A. et al., Anticancer research 29, 419-426 (2009); Farag, M. A. et al., Nature protocols 12, 1359-1377 (2017); Schulz, S. et al. Tetrahedron 60, 3863-3872 (2004); Blom, D. et al., Environmental microbiology 13, 3047-3058 (2011); Ahmed, W. M. et al., Analyst 143, 4155-4162 (2018); Leemans, M. et al., Biomarker Insights (2022)]. Challenges contributing to this gap include organism viability and material compatibility, exogenous contaminants, imaging capabilities, liquid-to-headspace ratio and signal sensitivity, gas flow characteristics and reproducibility, reusability, throughput, and adaptability for automation.

Here, the work addresses many of these gaps surrounding the analysis of VOCs from in vitro cultures through the design, development, and application of a custom borosilicate glass culture vessel (termed the “Biodome”) and imaging tray to demonstrate the advantages of the disclosed system or tool for biological analysis through the unbiased characterization of mammalian cell and bacterial volatilomes. Similar to a recently published approach [Franchina, F. A. et al., Journal of separation science 43, 1790-1799 (2020)], this work leverages the enhanced sensitivity, resolution, and separation capabilities of thermal desorption coupled to comprehensive two-dimensional gas chromatography with time-of-flight mass spectrometry (GC×GC-TOFMS) for metabolomic analysis [Keppler, E. A. et al., TrAC Trends in Analytical Chemistry 109, 275-286 (2018)]. This instrumentation was paired with an offline, open flow system designed to trap culture-derived VOCs using a tri-phase thermal desorption tube. The modular aspects of the disclosed system support “offline” culturing in a standard incubator and easy replacement of the input gas for adaptation to oxygen-sensitive organisms. The borosilicate glass culture vessel enables sterilization by autoclaving while also allowing for imaging directly through the device.

Given the intended biological applications, fluorescent imaging was first used to demonstrate that SK-OV-3 mammalian cell growth is equivalent to a standard T-75 flask. The exogenous VOCs originating from the flow system and culture media were extensively characterized in order to identify seven VOCs strictly observed in the SK-OV-3 ovarian adenocarcinoma in vitro volatilome, four of which have not been previously reported. Leveraging glycolysis-mediated isotopic labeling strategies [Lee, D. K. et al., ACS Central Science 4, 1037-1044 (2018)], 2-Decen-1-ol as cellularly derived with glucose as a carbon source was helped verified. Both in vitro and in vivo mammalian volatilomes typically show hydrocarbons as a major constituent. Therefore, to validate endogenous origin using an independent approach, RNA interference (RNAi) was applied to selectively knockdown lipid metabolism through silencing of CPT2 and identify at least four VOCs with evidence supporting biosynthesis derived from lipid metabolism. Finally, the functionality of the disclosed tool was extended by characterizing the Escherichia coli (E. coli) volatilome across the log and stationary phases of growth and identify six low abundance VOCs not previously observed in E. coli volatilome, with established pathways putatively giving rise to their biosynthetic origin. The work shown in this example demonstrates that the disclosed system or tool enables the reproducible sampling of complex biological matrices and provides sufficient sensitivity to allow for the identification of endogenous volatile metabolites and low abundance VOCs not present in the culture media or flow system.

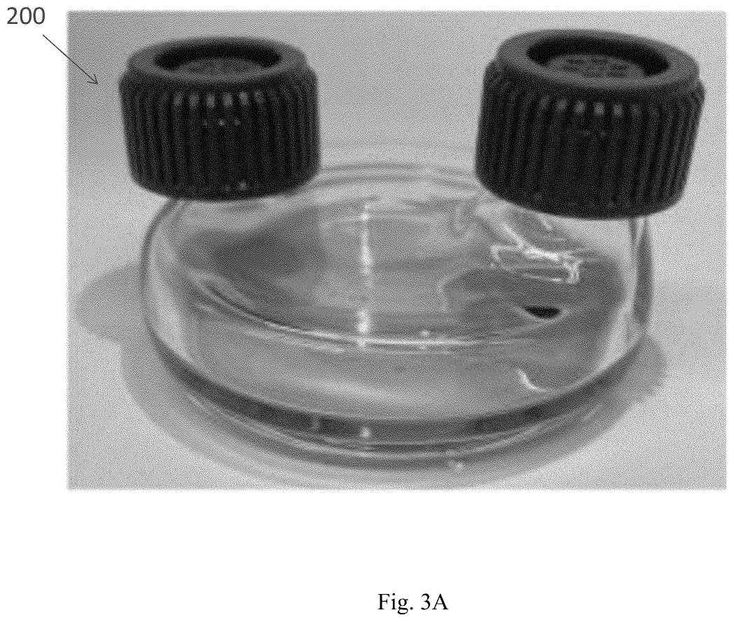

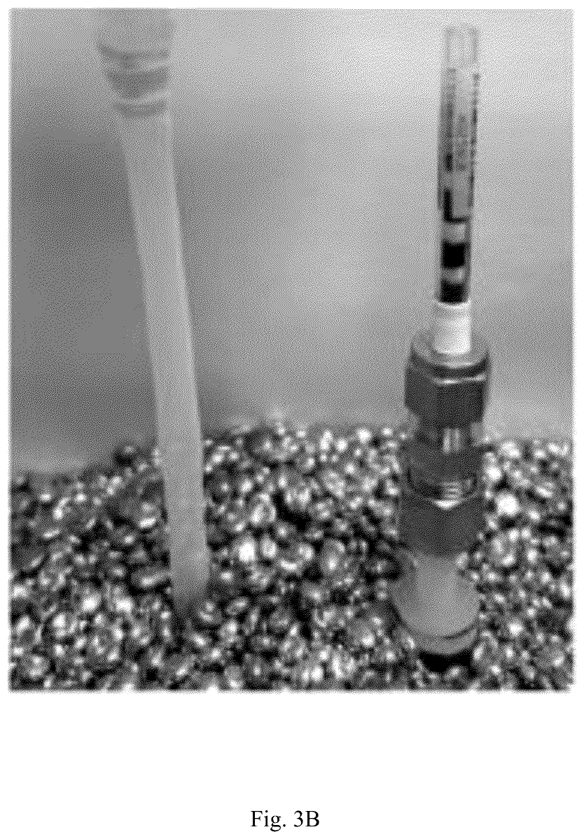

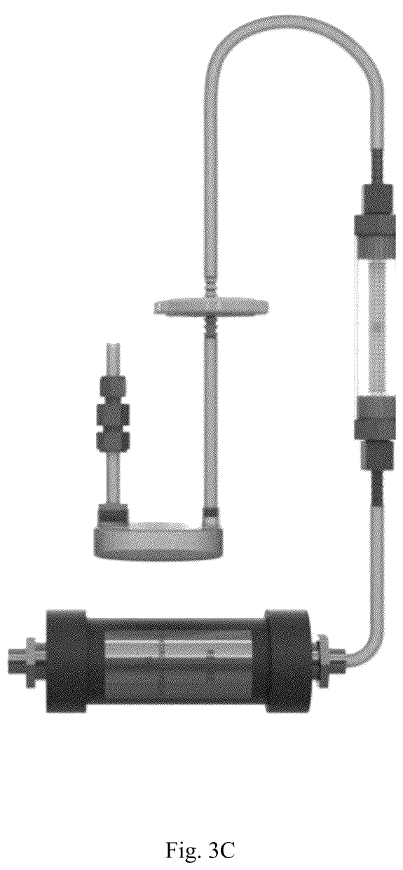

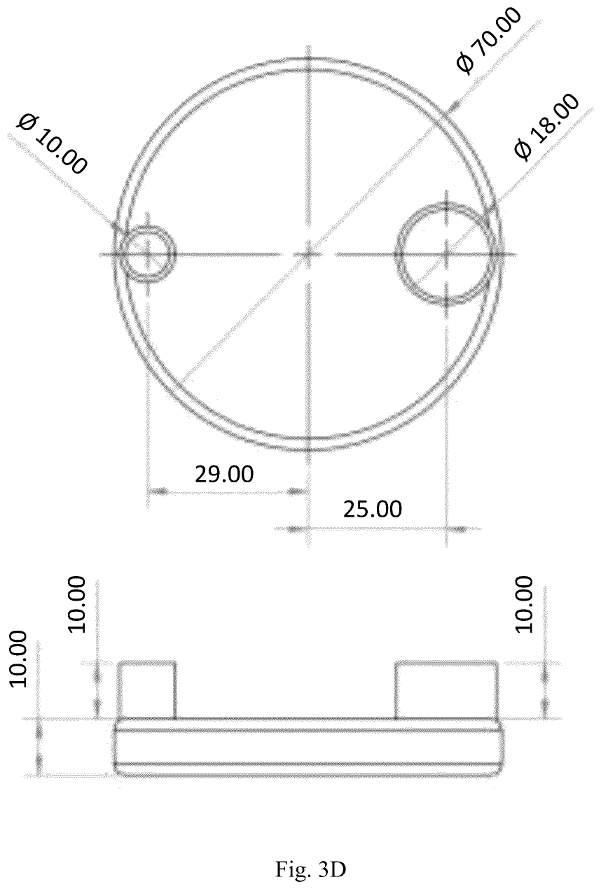

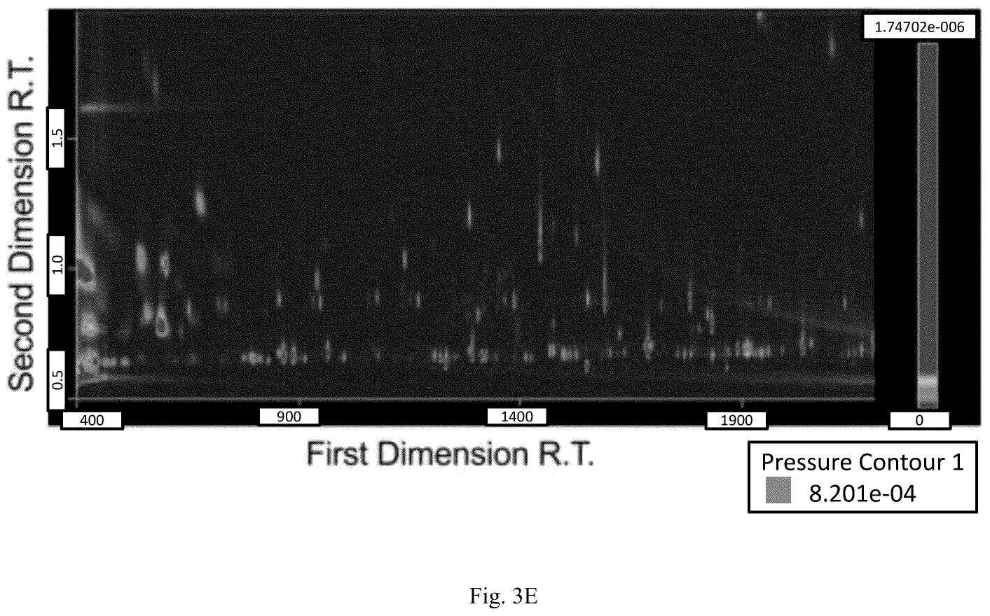

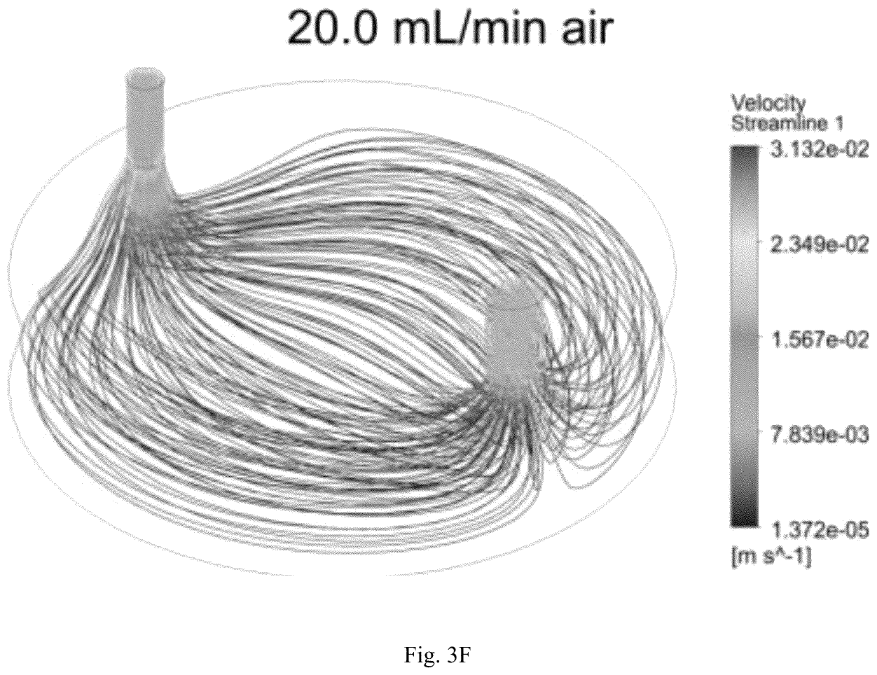

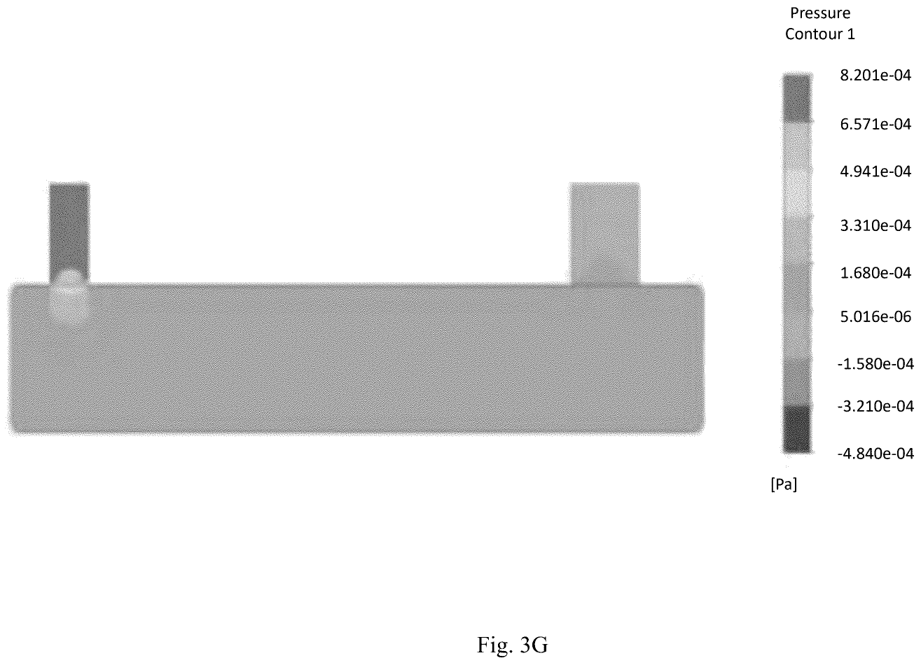

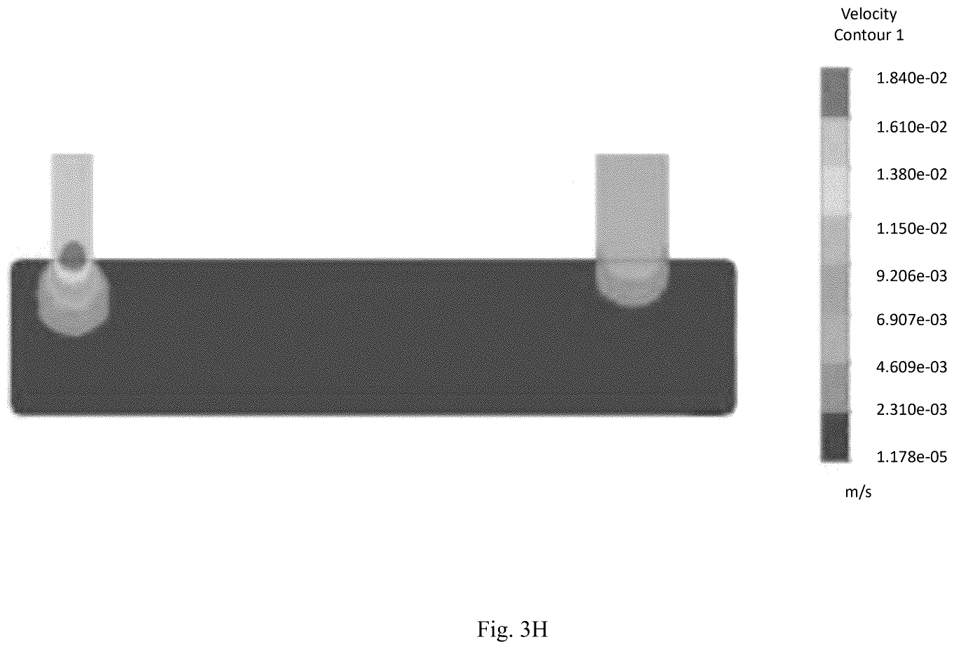

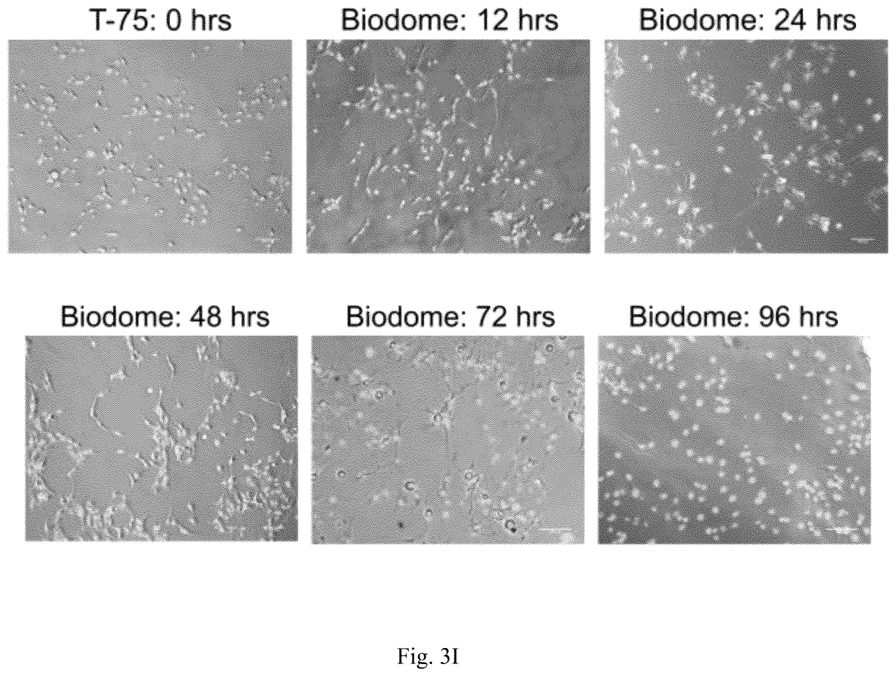

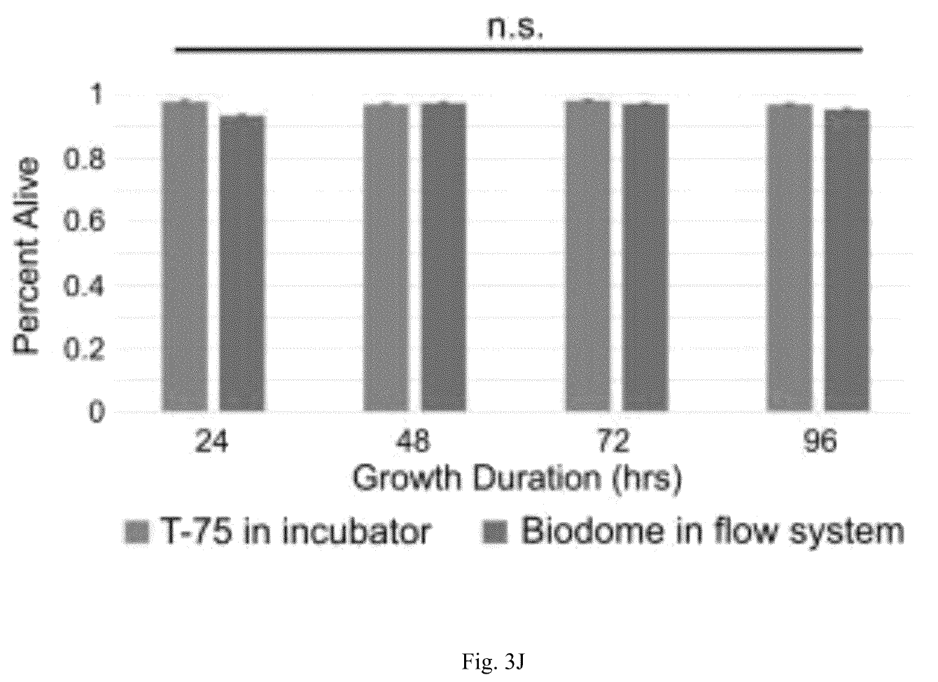

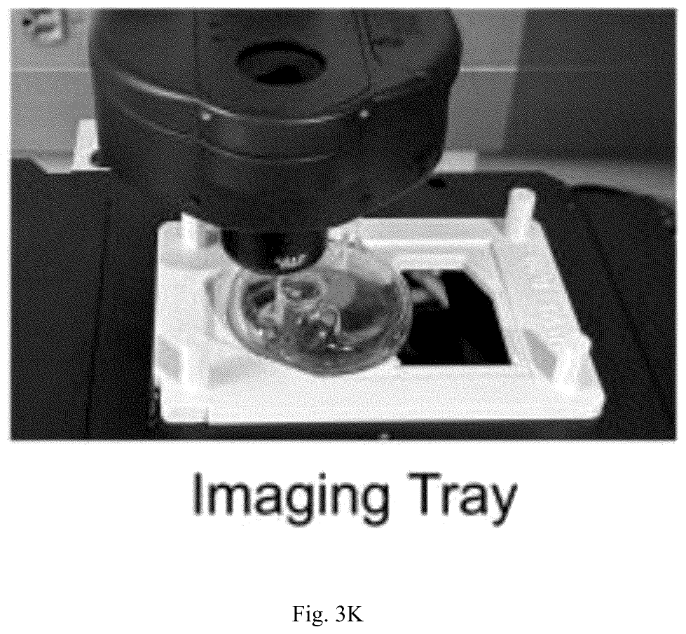

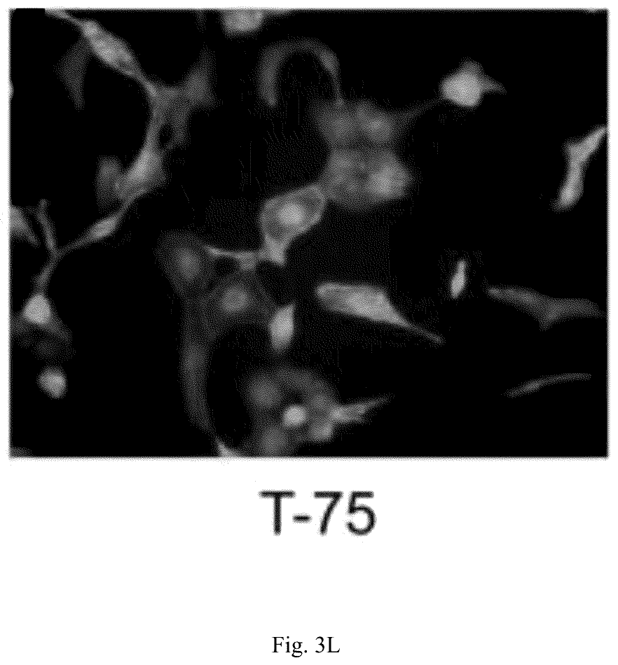

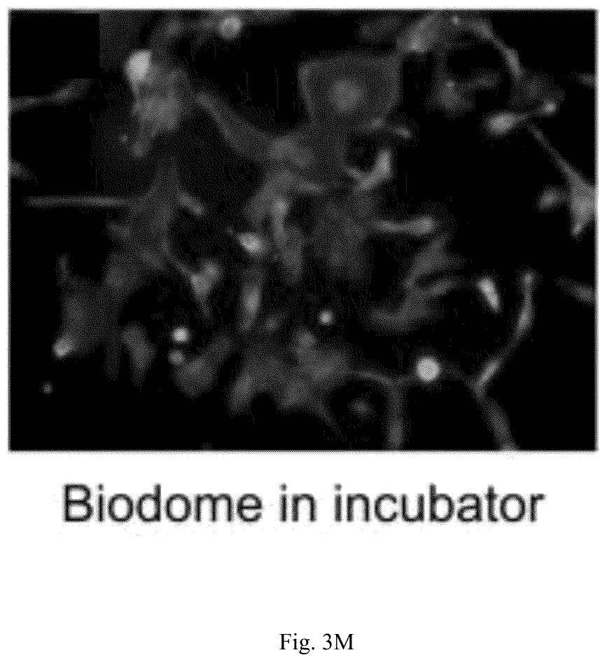

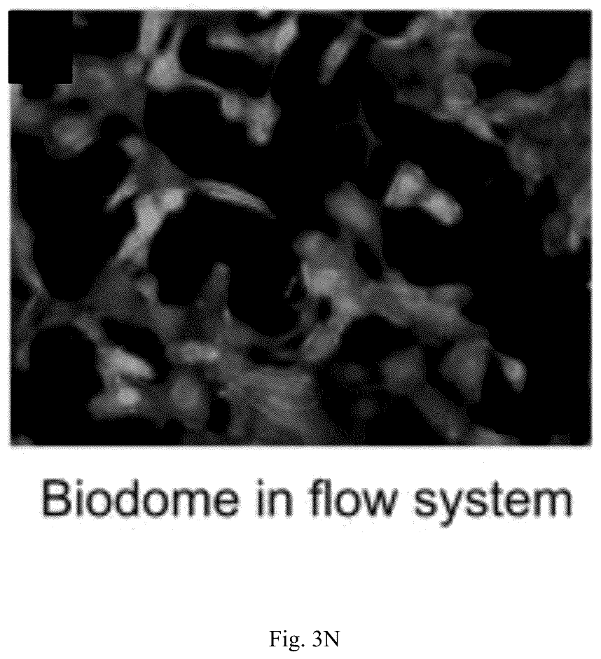

The biological VOC sampling system is comprised of a circular glass culture vessel, compressed gas cylinder and dual stage regulator, Nalgene™ 890 FEP tubing (Catalog #8050-0310, Thermo Scientific, Waltham, MA, USA), oxygen-compatible PTFE tape (Catalog #22485, Restek, Bellefonte, PA, USA), hydrocarbon trap (Catalog #22013, Restek), flow meter (Catalog #40402-0010, Riteflow, Bel-Art, Wayne, NJ, USA), sterile filter (0.2 m pore size, Pall Corporation, Port Washington, NY, USA), bead bath (LabArmor, Plano, TX, USA), Carbopack C, Carbopack B, and Carbosieve SIII sorbent thermal desorption tube (TDT; Gerstel, Linthicum, MD, USA), and inexpensive laboratory-made TDT adapter—incorporating a modular design to facilitate adoption for a variety of biological organisms (FIG. 3A to FIG. 3D). For example, the bead bath temperature can be raised to promote growth of thermophiles or replaced with an ice bath to allow study of psychrophiles. Similarly, the inflow gas can be replaced to support the analysis of oxygen-sensitive microorganisms. The system is designed to enable the continuous sampling of the headspace VOCs in the Biodome by flowing the desired compressed gas through the culture vessel, effectively increasing the cumulative VOC signal as compared to static equilibrium-based systems (i.e., solid phase microextraction (SPME) in a culture vessel or closed vial), as a consequence of Henry's Law.

FIG. 3A to FIG. 3N. show the design and development of the biodome. FIG. 3A shows the borosilicate glass culture vessel, termed the Biodome, containing 5 mL RPMI 1640 media. T-25 gas permeable flask caps were allowed to rest over the inlet and outlet of the vessel while in an incubator. FIG. 3B shows the placement of the Biodome during VOC sampling, showing the glass vessel is fully submerged in beads at 37° C. Also shown is the interface between the Biodome, TDT adapter, and TDT. A sterile rubber stopper and PTFE tape are used to create an airtight seal at the interface between the TDT and the adapter. FIG. 3C shows a 3D rendering showing the major components of the gas flow system including the hydrocarbon trap, flow meter, sterile filter, tubing, Biodome, TDT adapter, and TDT sampling tube. The compressed gas cylinder is not shown. FIG. 3D shows a schematic showing relevant dimensions (in mm; OD) for the Biodome culture vessel. FIG. 3E shows a representative GC×GC chromatogram spanning 400-2200 seconds in the first dimension and 0.5-2 seconds in the second dimension, following 24 hours of sampling SK-OV-3 ovarian adenocarcinoma cells. FIG. 3F shows fluid modeling in ANSYSM that demonstrates laminar flow conditions are maintained at flow rates≤20 mL/min (TDT adapter insert included). FIG. 3G shows the gauge pressure, and FIG. 3H shows velocity changes due to the inflowing gas are negligible at roughly half the Biodome height, limiting turbulent interactions with the surface of the cell culture media. FIG. 3I shows the results for a live/dead assay images in the Biodome showing viability across 0.5-4 days. A T-75 flask at t=0 (24 hours post seeding) is shown for reference. FIG. 3J shows the results for an independent live-dead assays performed using NucBlue™ (Hoechst 33342) and NucGreen™ ReadyProbes™ at 24-hour intervals, with live percentages estimated from 10 independent regions of the T-75 or Biodome culture vessels. No statistically significant differences in cell viability are observed demonstrating equivalence as compared to standard culture vessels. FIG. 3K shows a custom imaging tray designed in SolidWorks® 2019 and 3D printed to allow imaging using a Leica DMi8. FIG. 3L, FIG. 3M, and FIG. 3N show that typical SK-OV-3 morphology was visualized using fluorescent imaging and a live actin tracking stain (green color), Hoechst 33342 (blue color; live stain), and NucGreen (red color; dead stain), and no differences are observed using different culture vessels and growth environments. All brightfield and fluorescent images were taken using cells at passage<10.

The Biodome culture vessel is glass blown from borosilicate glass and features a 6.35 mm OD (¼ inch) inlet and 18 mm OD outlet (FIG. 3A, FIG. 3D), with heights equal to 10 mm. The internal chamber height also measures 10 mm, selected to enhance VOC sensitivity and reproducibility by optimizing surface area to volume ratio while limiting turbulent interactions between the inflowing gas and the surface of the liquid culture media. The thickness of the glass is roughly 1-2 mm and the diameter of the biodome is 70 mm, giving an approximate surface area of 38.5 cm². Vented caps from standard T-25 flasks were found to maintain a favorable environment for growth when allowed to rest over the inlet and outlet (FIG. 3A). Worth noting, no surface coating was required to promote cell adhesion and growth, increasing throughput, and reducing user burden.

The TDT adapter (FIG. 3B, FIG. 3C) is comprised of a size 3 silicon rubber stopper adjusted to a ˜15 mm diameter using a sterile razor (VWR, Radnor, PA, USA) with 6 mm hole introduced by a biopsy punch (Integra LifeSciences, Princeton, NJ, USA), Nalgene tubing (6.35 mm ID, 7.9375 mm OD, see above), one 6.35 mm (¼ inch) and one 7.9375 mm ( 5/16 inch) 316 stainless steel compression nut with metal ferrule (Swagelok Southwest Co., Phoenix, AZ, USA), and a 316 stainless steel 6.35 mm male to 7.9375 mm male adapter (Swagelok). For assembly of the adapter, see Methods. Although the initial design was intended for TDT sampling by two dimensional GC×GC-TOFMS (FIG. 3E), given the popularity of SPME for VOC analysis, the adapter was further modified in an inexpensive manner to enable this method by adding a 1 mL disposable syringe body (Electron Microscopy Sciences, Hatfield, PA), with the plunger removed, in line with the 6.35 mm (¼ inch) nut (replacing the TDT) and completely sealing the interface with oxygen-compatible PTFE.

To assess the gas flow characteristics through the headspace of the culture vessel, fluid modeling was performed in ANSYS Fluent® 2019 R3. Results show that laminar flow is maintained for gas flow rates≤20 mL/min, using the defined geometry (FIG. 3D, FIG. 3F to FIG. 3H). A flow rate of 11.7 mL/min was maintained for all testing, corresponding to a reading of 30 on the flow meter, unless otherwise noted. To enhance throughput, a multiplexed part was designed in SOLIDWORKS® 2019 and flow characteristics were assessed using ANSYS Fluent. Results show the part provides consistent flow rates at each of the three outlets, equal to 1/3 the total flow rate, while maintaining a single inlet. Given an internal chamber volume of approximately 200 cm³, the internal chamber can be flushed of environmental gas using a purge flow rate of 20 mL/min and a duration of 10 minutes.

Cell viability was considered in SK-OV-3 ovarian adenocarcinoma cells (HTB-77, ATCC, Manassas, VA) using live/dead fluorescence assays. 0.5×106 cells were seeded into the Biodome containing a total of 5 mL of RPMI1640 culture media containing 10% FBS and 1% Pen/Strep and given 24 hours to allow cell attachment. Brightfield images show time-dependent increases in Biodome confluency and high viability (FIG. 3I). A standard T-75 flask (VWR) 24 hours after seeding is shown for reference, equivalent to t=0 in the Biodome (FIG. 3I). Results from independent live/dead fluorescence assays (NucBlue and NucGreen ReadyProbes, Invitrogen, Carlsbad, CA) show no significant differences in SK-OV-3 cell viability in the Biodome, as compared to a standard T-75 flask in a humidified incubator, for a minimum duration of 4 days (FIG. 3J). Cell viability through morphological fluorescence staining was further considered using a live actin tracking stain (CellMask, Invitrogen). To enable imaging on a Leica DMi8 microscope, a custom imaging tray was designed in SOLIDWORKS® 2019 (FIG. 3K). Following seeding in the appropriate culture vessel, SK-OV-3 cells were allowed to proliferate for 48 hours before staining, and the results show that cell morphology in the Biodome is comparable to a T-75 flask grown in a standard incubator (FIG. 3L to FIG. 3N). Taken together, the findings demonstrate that the Biodome tool maintains favorable growth conditions for at least 4 continuous days, without the need for surface treatment.

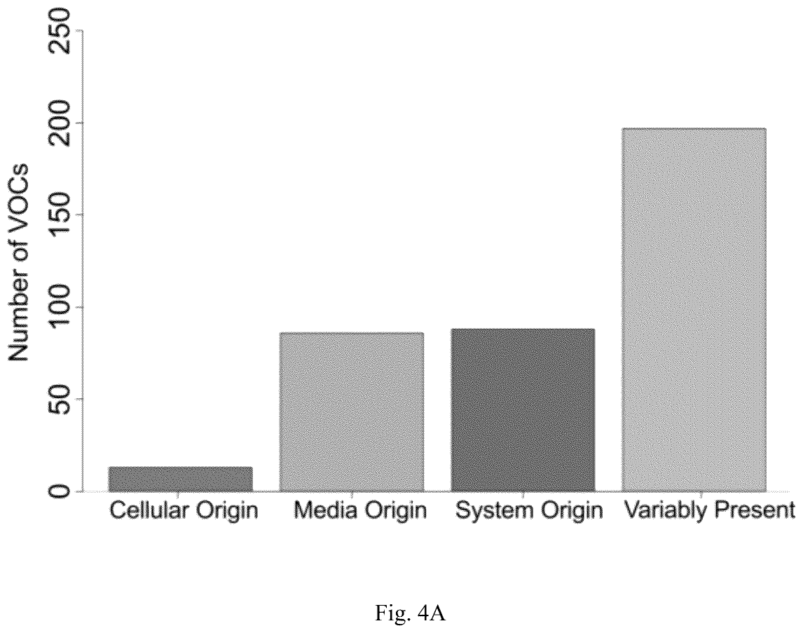

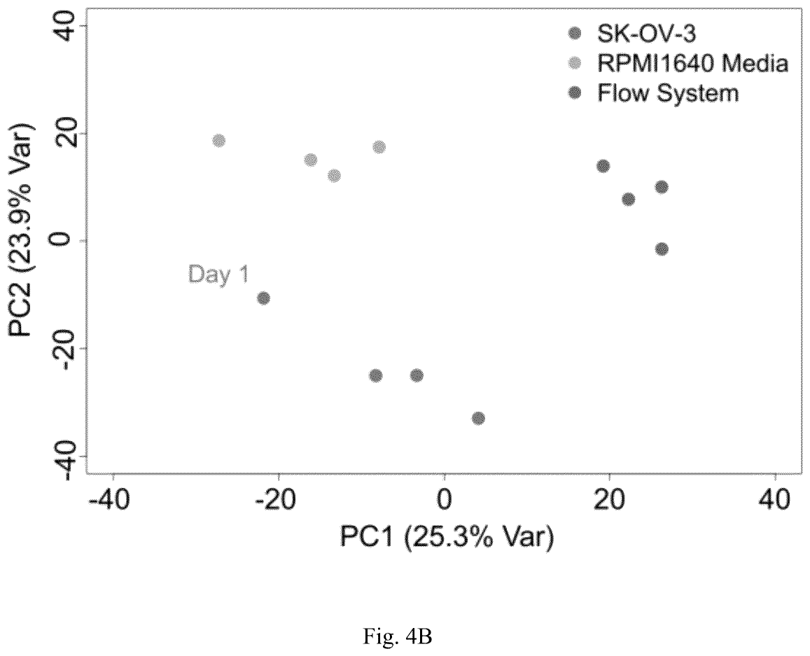

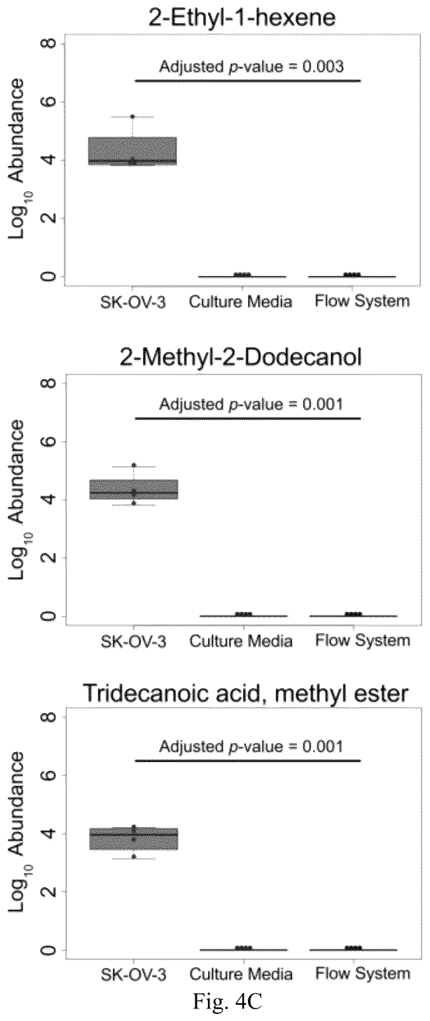

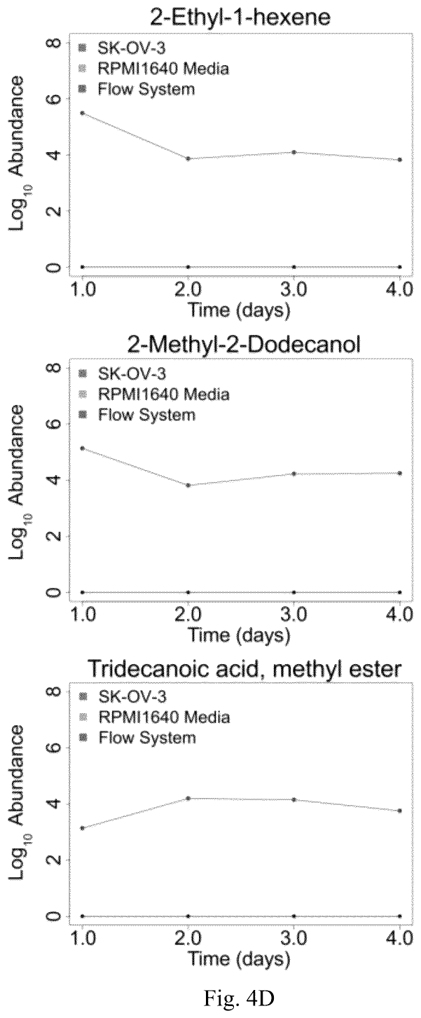

FIG. 4A through FIG. 4D shows the volatilome characterization using the in vitro Biodome sampling tool. FIG. 4A shows a bar chart showing the number of VOCs detected, categorized according to their proposed origin. Variably present indicates the VOCs were observed inconsistently in two or more experimental conditions. FIG. 4B shows a PCA plot showing distinct clusters when considering the first two principal components, using the 384 features comprising the filtered volatilome. The first day SK-OV-3 volatilome was seen to resemble the media control more closely, as compared to the other three days, suggesting a 24 “flushing” period prior to collection may help with the reproducibility of in vitro mammalian volatilomes. FIG. 4C shows representative boxplots, and FIG. 4D shows line plots showing three low-abundance VOCs (ID<4) reproducibly and exclusively detected in the SK-OV-3 ovarian adenocarcinoma in vitro volatilome. Abundance values were calculated by integrating the peak area using the unique mass and log₁₀ transformed. The adjusted p value was calculated using a two-tailed student's t-test and corrected using the Benjamini-Hochberg procedure [Benjamini, Y., et al., Journal of the Royal statistical society: series B (Methodological) 57, 289-300 (1995)].

SK-OV-3 cells (human origin) were seeded into the Biodome glass culture vessel and allowed to adhere and proliferate for 24 hours. Prior to sampling, culture media was replaced to ensure the accumulation of VOCs were minimized. Based on the previously observed growth characteristics (FIG. 3H to FIG. 3L), cells were subject to VOC collection for 4 continuous days with the TDT replaced every 24 hours, although shorter sampling times were also shown to be viable. RPMI 1640 was maintained as the primary medium, however dialyzed FBS was implemented in place of FBS, supported by a previous work that demonstrated serum-free growth media is favorable for VOC analysis [Yamaguchi, M. S. et al., Journal of Chromatography B 1090, 36-42 (2018)].

Results show the Biodome system supports the identification of cellular VOCs absent in the flow system and culture vessel, both with and without culture media. To focus on the reproducible aspects of the cellular and control volatilomes, features that were removed were observed in fewer than 3 of the 4 days of sampling, resulting in a total of 384 unique peaks (FIG. 4A). The filtered volatilome was subsequently subject to principal component analysis (PCA) and distinct clusters corresponding to the control and SK-OV-3 conditions were observed (FIG. 4B).

Roughly 22% (86/384) of the unique features were observed in all samples (allowing for one missing observation), including the empty Biodome control, suggesting these are inherent contaminants associated with the use of the in vitro tool (FIG. 4A). Further, it was found that five VOCs capture 9-44% of the total chromatographic signal in the empty culture vessel, after removing poorly resolved peaks with a first-dimension retention time<400 seconds, as further discussed herein. These high abundance contaminant VOCs (naming confidence ID level≤3 [Sumner, L. W. et al., Metabolomics 3, 211-221 (2007)]) were found to include: Ethyl acetate, Pyridine, 1-Methyl-2-pyrrolidinone, 3,7-Dimethyl-1-octene, and 1-Dodecanol. An alkane standard was run to allow identification of VOC retention indices (RI) and used to support name assignment where possible. A further 13% (50/384) of the unique features were observed in both the culture media and SK-OV-3 experimental conditions but not the empty Biodome control, suggesting media origin (FIG. 4A). Interestingly, 38 VOCs (9.9%) were reliably detected in the media volatilome but not the SK-OV-3 volatilome, which may indicate SK-OV-3 metabolic biotransformation but more likely signal suppression, given 73.7% had a median signal-to-noise ratio<150:1. Lastly, a total of 13 VOCs (3.4%) were strictly observed in the experimental condition containing SK-OV-3 cells, allowing for 1 total spurious peak from controls. The remaining features (n=197) that passed the filtering criteria were primarily present in: (i) system control (122; 61.9%), (ii) media control (39; 19.8%), or (ii) SK-OV-3 condition (36; 18.3%) yet were variably present in the other two experimental groups, limiting further interpretation. Four of these VOCs were strictly observed in cellular volatilomes that have not been previously detected in mammalian cancer cells (2-Ethyl-1-hexene; 2-Methyl-2-dodecanol; Tridecanoic acid, methyl ester; and Dodecyl acrylate also shown as bolded names in Table 1), emphasizing the enhanced sensitivity afforded by the in vitro Biodome tool and imaging tray (Table 1; FIG. 2C, FIG. 2D, FIG. 3K).

Cell-specific VOCs were primarily hydrocarbons but spanned multiple chemical classes, including the reproducible detection of three VOCs that fell below the naming threshold. Despite the lower naming confidence, mass spectral data could be used to support their identification as two alkanes eluting at 2336 and 2355 seconds, and an alkene eluting at 2569 seconds, in the first dimension (Table 1). Importantly, while mass spectra indicate alkane (m/z 43, 57, 71, 85, etc.) and alkene (m/z 41, 55, 69, 83, etc.) backbones, second dimension retention times suggest they may be functionalized. Of the VOCs observed strictly in the SK-OV-3 volatilomes, over half (54%) had a median signal-to-noise ratio<150:1, emphasizing the capability of the approach to recover low abundance volatiles that may be missed using equilibrium-based sampling approaches.

TABLE 1
VOCs reproducibly detected from SK-OV-3 cells

| | | | SK-OV-3 vs | | | | |
|---|---|---|---|---|---|---|---|
| | | SK-OV-3 vs | System | | | | |
| | | Media FDR | Control FDR | | | | |
| | Chemical | adjusted p- | adjusted p- | Avg | Avg | | ID |
| VOC | Class | value | value | 1tR (s) | 2tR (s) | RI | Level |
| 2-Methylhexane | Hydrocarbon | 0.07836 | 0.00912 | 605 | 0.63 | 634* | 3 |
| 1-Butanol | Alcohol | 0.14659 | 0.07836 | 724 | 1.11 | 699* | 2 |
| 3-Methylbutanal | Aldehyde | 0.14155 | 0.07836 | 731 | 0.8 | 703* | 2 |
| Unknown 1 | | 0.27552 | 0.07836 | 869 | 1.14 | 808 | 4 |
| 2-Ethyl-1-hexene | Hydrocarbon | 0.00303 | 0.00303 | 922 | 0.69 | 832 | 3 |
| 2,4,6- | Hydrocarbon | 0.07836 | 0.21699 | 1047 | 0.66 | 889 | 3 |
| Trimethylheptane | | | | | | | |
| 1,2,3- | Aromatic | 0.00306 | 0.07836 | 1299 | 0.89 | 1005 | 2 |
| Trimethylbenzene | Hydrocarbon | | | | | | |
| 2-Methyl-2- | Alcohol | 0.00131 | 0.00131 | 2238 | 1.29 | 1530 | 3 |
| dodecanol | | | | | | | |
| Hydrocarbon 1 | Hydrocarbon | 0.00099 | 0.00099 | 2336 | 1.98 | 1595 | 4 |
| Hydrocarbon 2 | Hydrocarbon | 0.0012 | 0.0012 | 2355 | 1.45 | 1606 | 4 |
| Tridecanoic acid, | Ester | 0.00131 | 0.00131 | 2537 | 1.33 | 1710* | 2 |
| methyl ester | | | | | | | |
| Dodecyl acrylate | Other | 0.0012 | 0.0012 | 2568 | 1.02 | 1724* | 2 |
| Alkene 1 | Hydrocarbon | 0.00131 | 0.00131 | 2569 | 1.25 | 1724* | 4 |

Within Table 1, the compound name is presented first, followed by the functional group class. Bolded names indicate that the VOC was not detected in the media or system controls and have not been previously reported in vitro. The adjusted p value was calculated using a two-tailed student's t-test, comparing the control and SK-OV-3 volatilomes post-filtering. The first (¹t_{R}) and second (²t_{R}) dimension retention times are included, with the retention index calculated using a C8-C20 alkane standard mix. Finally, the VOC naming confidence level is reported according to previously established standards.

Stable isotope labeling of metabolites in cell culture (SILMC): For the global and unbiased assessment of endogenous VOC production [Lee, D. K. et al., ACS Central Science 4, 1037-1044 (2018)], SK-OV-3 cells were passaged 20 times supplemented with ¹³C₆-D-glucose (Cambridge Isotope Laboratories, Tewksbury, MA, USA) to isotopically label glucose-derived metabolites (FIG. 5A). As described previously, labeled (¹³C₆-D-glucose) and unlabeled (¹²C₆-D-glucose) cells were seeded into the Biodome device and allowed to grow out for 24 hours.

Labeled and unlabeled SKOV-3 lines, at equivalent passage number, were subject to VOC collection for 4 continuous days, with TDU tubes replaced every 24 hours (FIG. 5A). TDU tubes were stored for a maximum of 8 days at 4° C. to limit degradation [Harshman, S. W. et al., Journal of breath research 10, 046008 (2016)] prior to analysis by GC×GC-TOFMS. Chromatograms from the same experimental condition were aligned using ChromaTOF®, and subsequently processed using custom R code to match isotopically labeled peaks to unlabeled ones using thresholding of the first- and second-dimension retention times—given ¹³C-labeled VOCs should elute from the column at the same time as 12C VOCs [Berg, T. et al., Journal of Chromatography A 1344, 83-90 (2014); Kempa, S. et al., Journal of basic microbiology 49, 82-91 (2009)]. Following post-processing in R, putative ¹³C-labeled VOCs were manually validated by comparing ions and parent fragments between the ¹²C and ¹³C experimental conditions.

FIG. 5A through FIG. 5E shows glycolysis-mediated isotopic labeling in the Biodome. FIG. 5A shows a diagram showing the workflow implemented for the stable isotope labeling of glycolysis-derived VOCs. Representative mass spectrums comparing (FIG. 5B) labeled and unlabeled, (FIG. 5C)¹³C-labeled and media control, and (FIG. 5D) ¹³C-labeled and flow system control were selected using the peak with the highest signal-to-noise ratio across the four days of sampling. Despite observing 2-Decen-1-ol in non-cellular control volatilomes, many low intensity fragments remained uniquely observed in the ¹³C-labeled spectra. Breakout images showing m/z ranges between 107-114, 122-128, and 137-147 amu were generated in R using raw mass spectral data. The proposed ¹³C-labeled structure is indicated in red, while annotations correspond to the unlabeled (¹²C) fragments. Fragments without an annotation are possibly derived from secondary bond rearrangements. FIG. 5E shows a proposed metabolic pathway for ¹³C incorporation into 2-Decen-1-ol.

The results capture one VOC, 2-Decen-1-ol, with mass spectral information supporting 13C-labeling derived from ¹³C6-glucose (FIG. 5B; Table 2).

Possible structures are provided for mass fragments primarily or uniquely detected in the ¹³C-labeled spectra (Table 2). Further evidence supporting the claims include, (i) chromatographic elution of labeled and unlabeled 2-Decen-1-ol was consistent, with a relative standard deviation<1% in the first- and second-dimension, (ii) the mid-polar retention index falls within the expected range for 2-Decen-1-ol, leading to a naming confidence level of 2, and (iii) 2-Decen-1-ol has been previously observed to act as an endogenous metabolite in humans and observed extracellularly [Wishart, D. S. et al., Nucleic acids research 50, D622-D631 (2022)]. Despite observing 2-Decen-1-ol in media and flow system control volatilomes, many tentative ¹³C fragments remained uniquely observed in the ¹³C-labeled SK-OV-3 spectra (FIG. 5C, FIG. 5D). Analyte fragments of m/z 103, 107, 114, 125, 126, 127, 133, 141, 142, 143 and 147 were strictly observed in the ¹³C-labeled spectra, corresponding to the addition of 2 carbon isotopes near the alcohol functional group, with spectral and metabolic evidence supporting carbon positions 1 and 2 (FIG. 5E).

Importantly, the mass spectral data indicates variable ¹³C labeling, which may be a consequence of the inherently low ¹³C-labeled fraction and/or multi-step biosynthesis routes, with detectable mass shifts between 1-6 amu depending on the sampling day (Table 2). Furthermore, a ¹³C-shifted parent ion beyond an m/z of 156 was not observed, however given the parent peak had a median relative intensity of 0.5% across all spectra, it is likely that any labeled fraction fell below the detectable limit. To enhance detectable isotope fraction, future studies may benefit from using multiple ¹³C-labeled carbon sources (e.g. L-glutamine) [Wishart, D. S. et al., Nucleic acids research 50, D622-D631 (2022)].

TABLE 2
Putative ¹³C fragments of 2-Decen-1-ol

| Isotope | Relative Intensity | Relative Intensity | |
|---|---|---|---|
| Fragment (amu) | in C-labeled (%) | in C-labeled (%) | Possible Structure(s) |
| 59 | 0.7, 1.1, 0.0, 0.0 | 0.5, 0.2, 0.4, 0.5 | ¹²C₁¹³C₂H₅O |
| 91 | 0.6, 0.0, 0.8, 0.0 | 0.3, 0.2, 0.2, 0.2 | |
| 100 | 0.0, 0.0, 0.0, 0.0 | 0.1, 0.0, 0.0, 0.2 | ¹²C₆¹³C₁H₁₅; ¹²C₅¹³C₁H₁₁O |
| 103 | 0.0, 0.0, 0.0, 0.0 | 0.2, 0.0, 0.0, 0.0 | ¹²C₂¹³C₄H₁₅; ¹²C₂¹³C₄H₁₁O |
| 107 | 0.0, 0.0, 0.0, 0.0 | 0.1, 0.0, 0.2, 0.0 | |
| 114 | 0.0, 0.0, 0.0, 0.0 | 0.1, 0.0, 0.0, 0.0 | ¹²C₆¹³C₁H₁₁O |
| 125 | 0.0, 0.0, 0.0, 0.0 | 0.3, 0.0, 0.2, 0.3 | ¹²C₉H₁₆ |
| 126 | 0.0, 0.0, 0.0, 0.0 | 0.1, 0.0, 0.0, 0.0 | ¹²C₈¹³C₁H₁₆ |
| 127 | 0.0, 0.0, 0.0, 0.0 | 0.1, 0.0, 0.0, 0.0 | ¹²C₇¹³C₂H₁₆ |
| 133 | 0.0, 0.0, 0.0, 0.0 | 0.3, 0.0, 0.0, 0.0 | ¹²C₂¹³C₆H₁₅O |
| 139 | 0.0, 1.3, 0.0, 0.0 | 0.2, 0.4, 0.0, 0.2 | ¹²C₁₀H₁₉ |
| 141 | 0.0, 0.0, 0.0, 0.0 | 0.4, 0.0, 0.0, 0.5 | ¹²C₈¹³C₂H₁₉; ¹²C₉H₁₇O |
| 142 | 0.0, 0.0, 0.0, 0.0 | 0.1, 0.3, 0.0, 0.0 | ¹²C₇¹³C₃H₁₉; ¹²C₈¹³C₁H₁₇O |
| 143 | 0.0, 0.0, 0.0, 0.0 | 0.1, 0.0, 0.0, 0.0 | ¹²C₆¹³C₄H₁₉; ¹²C₇¹³C₂H₁₇O |
| 147 | 0.0, 0.0, 0.0, 0.0 | 0.2, 0.0, 0.0, 0.0 | |

As shown in Table 2, Isotope fragments are presented first increasing in mass, followed by the relative intensity in the unlabeled (¹²C) and ¹³C-labeled 2-Decen-1-ol mass spectra for all four days of sampling. Chemical structures potentially giving rise to the observed signal are proposed where possible, although secondary rearrangements cannot be ruled out. Further, mass fragments without a listed structure are reasoned to be derived from secondary bond rearrangements.

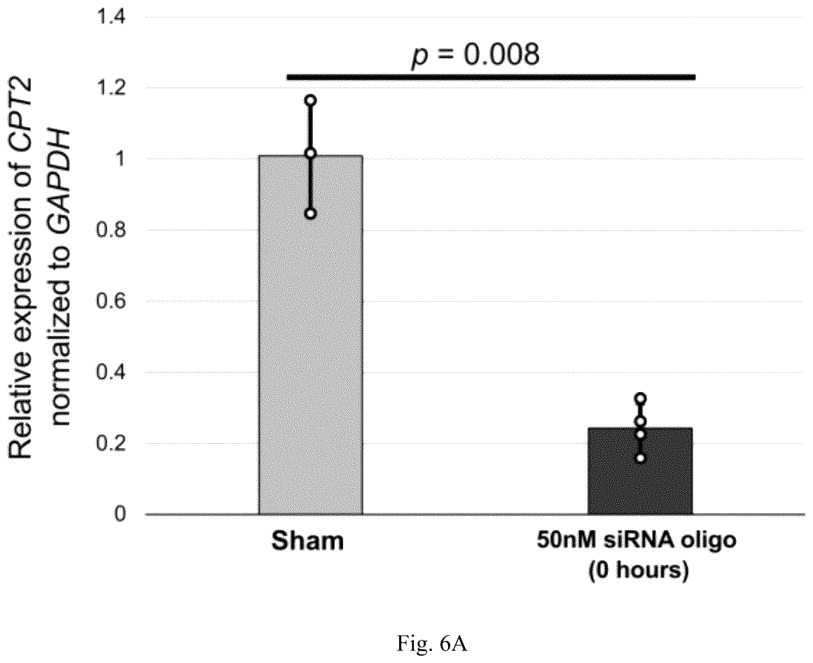

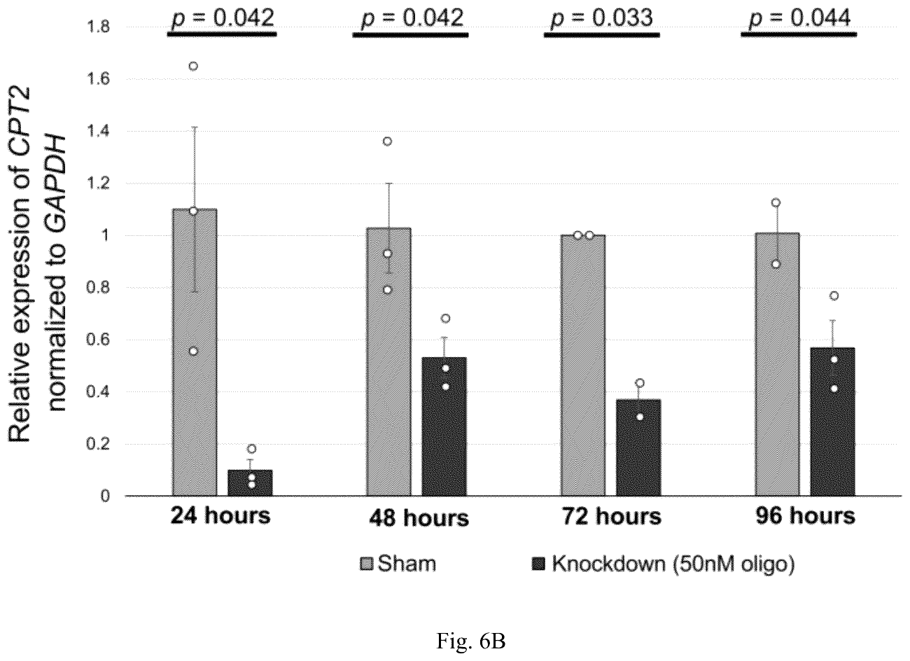

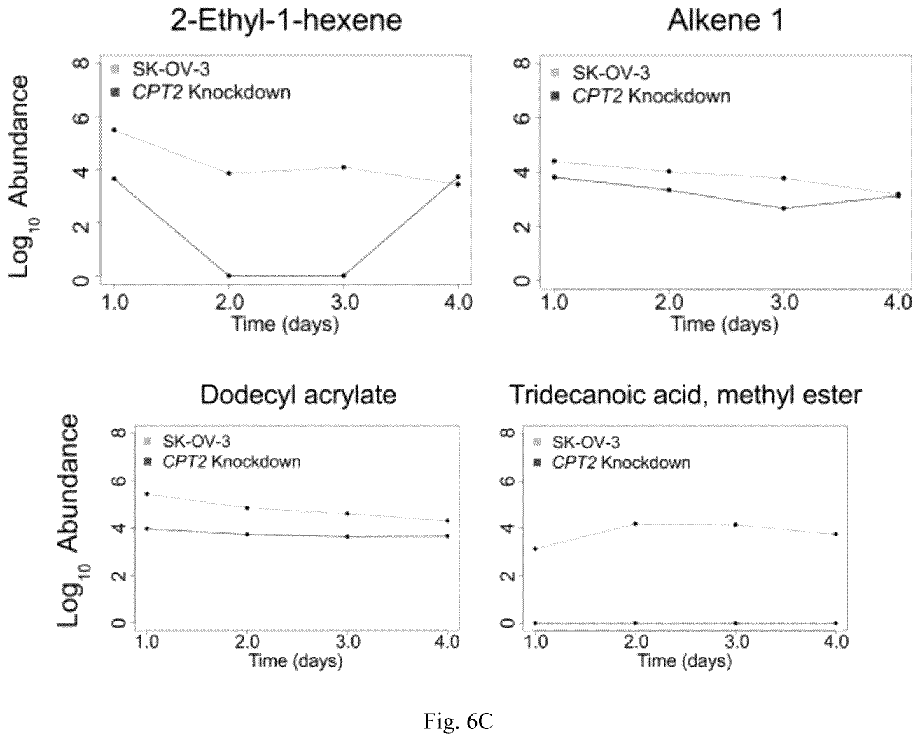

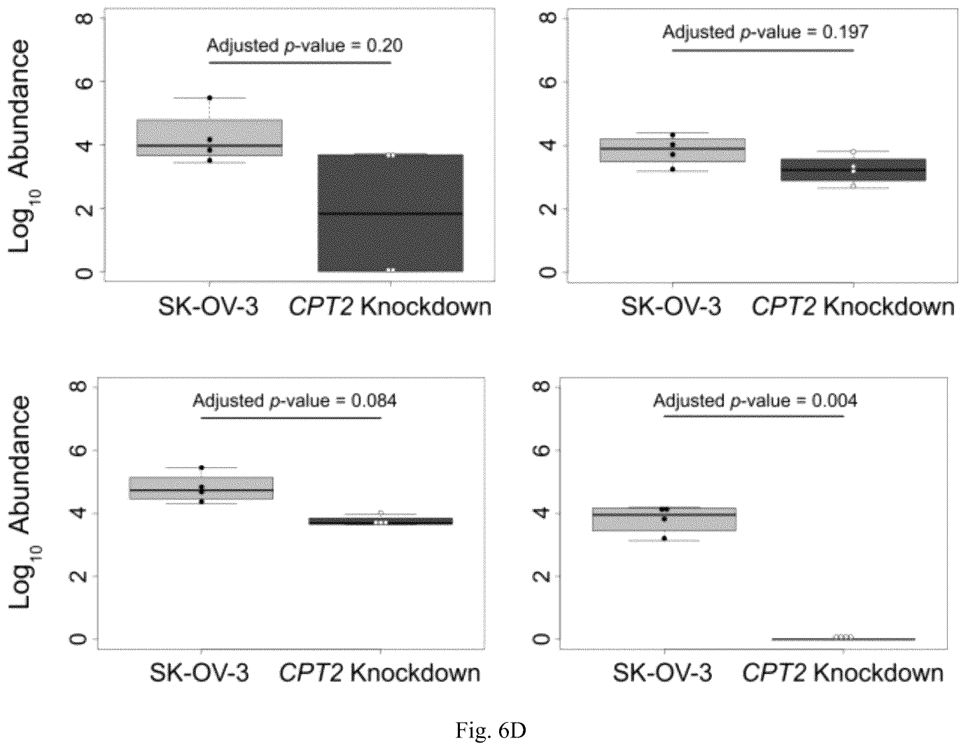

Gene knockdown supports endogenous production of cellular VOCs: To highlight the viability and analyte sensitivity benefits of in vitro volatolomics in the Biodome, the relationship between the biosynthetic origin of cellular VOCs and CPT2 transcriptional levels in SK-OV-3 cells were investigated. The CPT2 expression levels were altered by treating the SK-OV-3 cells with pooled siRNA oligonucleotides mediating gene knockdown. The expression levels of the CPT2 relative to the housekeeping gene GAPDH was determined using RT-qPCR. Results show CPT2 expression was significantly reduced in siRNA-treated SK-OV-3 cells to 24.3±7.01% (mean±S.E.M; p<0.05) at t=0 hrs, indicating successful transfection and gene knockdown (FIG. 6A). The expression levels of CPT2 in the transfected SKOV-3 cells were also monitored in a 6-well plate format over a 96-hour time period. As depicted in FIG. 6B, the CPT2 expression level during the initial 24 hours showed a significant decrease to 9.83±4.20% (p<0.05). Subsequently, after 48 hours, the expression levels increased to 53.0±7.82% (p<0.05). Following 72 hours, the expression levels decreased to 36.8±6.49% (p<0.05) and were recorded as 56.8±10.5% (p<0.05) after 96 hours.

FIG. 6A through FIG. 6D shows knockdown of CPT2 supports endogenous VOC production. FIG. 6A shows the expression of CPT2 quantified by RT-qPCR at the start of VOC collection (time=0) using biologically independent replicates. FIG. 6B shows the expression of CPT2 was independently mapped across the four days of VOC sampling following siRNA-mediated knockdown in SK-OV-3 cells at equal passage number using biologically independent replicates. FIG. 6C shows four VOCs not found in controls showing decreased abundance is consistent with the transient inhibition of β-oxidation via CPT2 knockdown, across 4 days of measurement. FIG. 6D shows boxplots showing significant or trending toward significant decreases in endogenous VOC abundance following integration by unique mass and log₁₀ transformation. P-values were adjusted using the Benjamini-Hochberg procedure [Benjamini, Y. et al., Journal of the Royal statistical society: series B (Methodological) 57, 289-300 (1995)].

Associated with the knockdown of CPT2 in SK-OV-3 transfected cells, at least four VOCs were found to have decreased abundances consistent with the transient inhibition across the 96-hour time period (FIG. 6C, FIG. 6D). In the case of 2-Ethyl-1-hexene, the median decrease in raw peak abundance was found to be 77%, with a roughly 98.5% decrease observed during the first day of sampling (FIG. 6C, FIG. 6D). Similarly, the unknown alkene abundance was observed to have a median decrease of 78.8%, with a 74% reduction on the first day, followed by 79%, 92%, and 15% on days 2, 3, and 4 respectively. Dodecyl acrylate showed a 91.1% decrease in the median abundance following knockdown of CPT2 with reductions of 97%, 92%, 89%, and 77% at days 1-4, respectively. Observations at each time point are consistent with the transient recovery of CPT2 following siRNA-mediated knockdown suggesting this VOC is directly linked to lipid β-oxidation (FIG. 6B, FIG. 6C). Although tridecanoic acid, methyl ester was not observed in the CPT2 knockdown volatilome, this VOC was determined to have low abundance with a median signal-to-noise ratio of 30 across the four days of sampling. The data processing method set a signal-to-noise threshold at 5, therefore the greatest reduction in signal while still allowing detection would be ˜85%, suggesting that the lack of observation may still support association with lipid metabolism given the inherently low abundance. Although signal-to-noise thresholding may partially explain the difference in reported abundance, a significant decrease in tridecanoic acid, methyl ester (adjusted p=0.004) and a trend towards significance (adjusted p=0.084) in the case of dodecyl acrylate after correction for multiple hypothesis testing were observed. Interestingly, production of dodecyl acrylate and the alkene were consistent in SK-OV-3 cells despite knockdown of CPT2, with the highest amount of signal detected on the first day, suggesting these VOCs may be associated with cellular growth and proliferation.

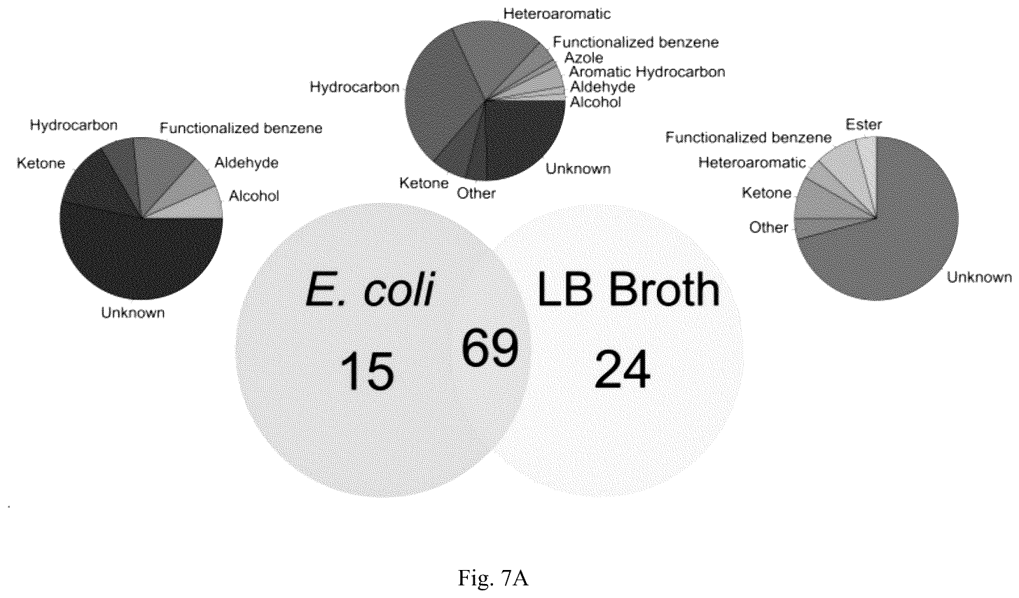

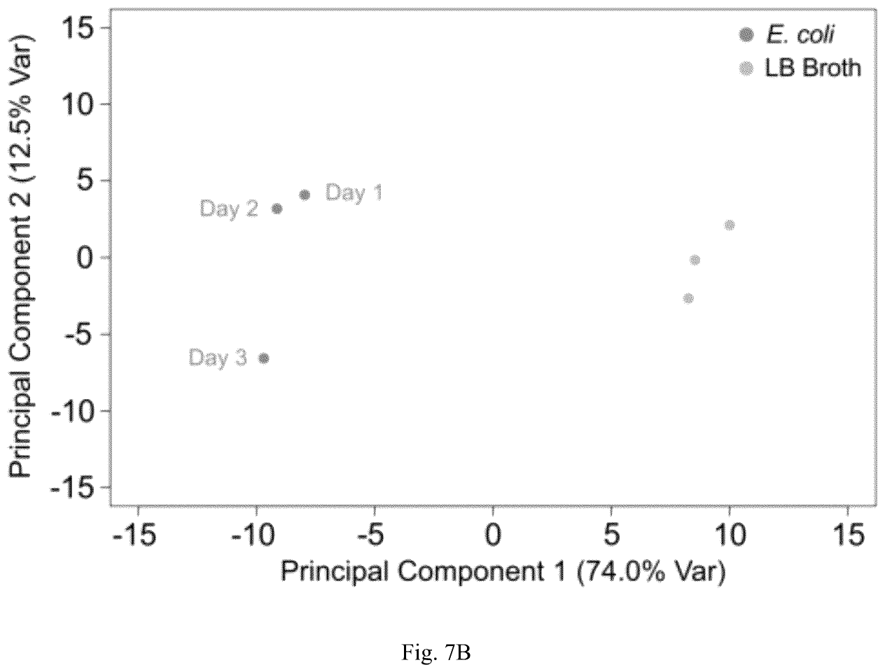

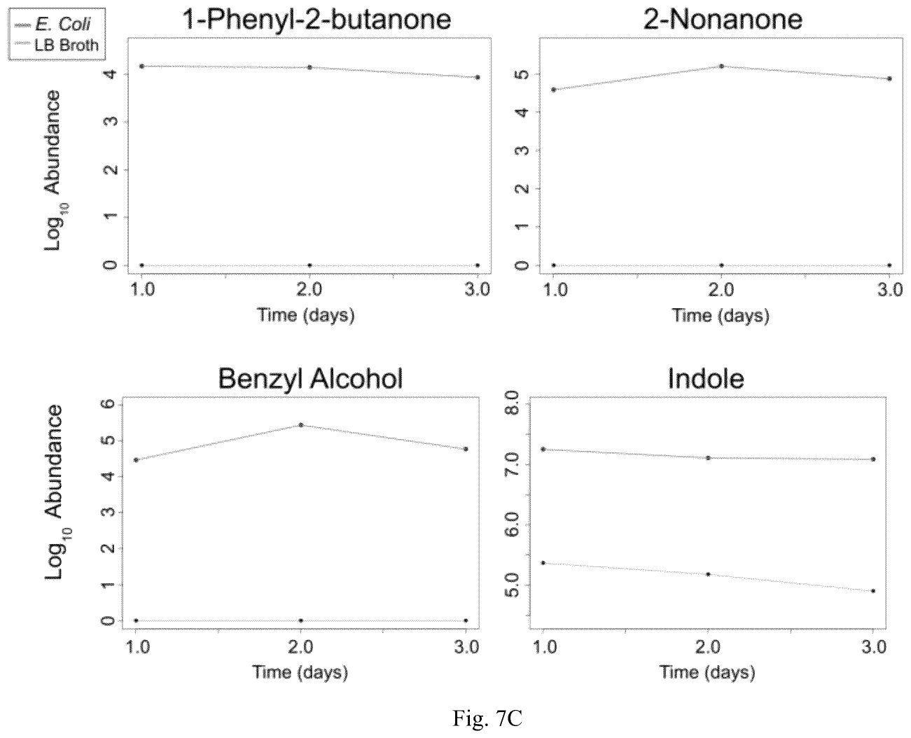

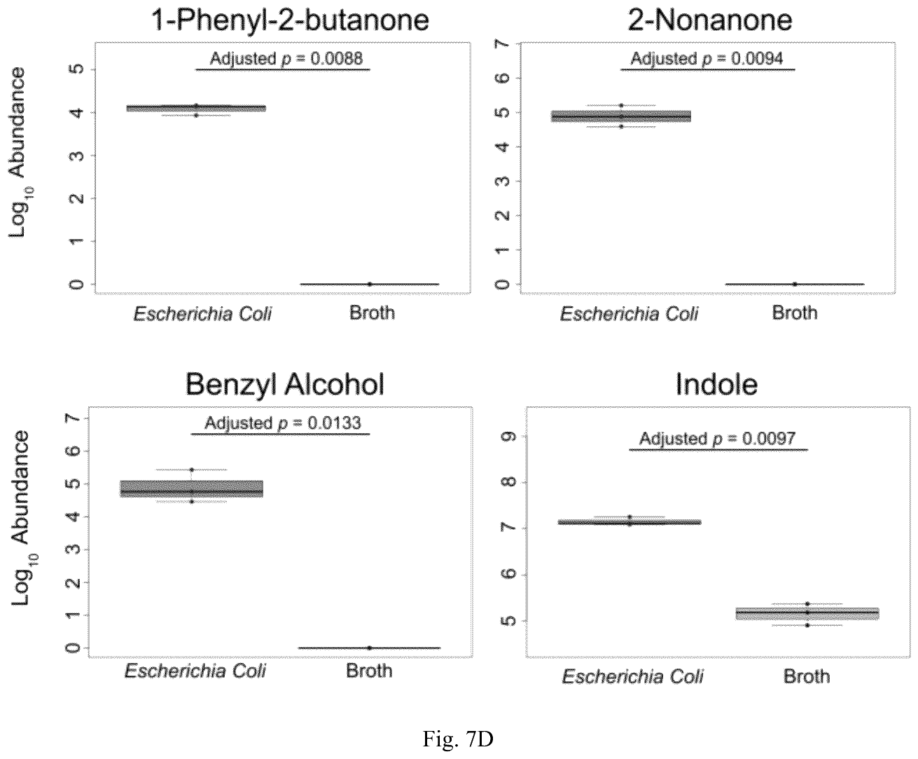

Bacterial adaptation: To extend the functionality of the system or tool, VOCs originating from Escherichia coli, strain DH5α, analyzed across 3 days, during the log and stationary phases of growth. E. coli were transformed with plasmid containing ampicillin resistance gene and seeded into the Biodome containing LB Broth and 100 g/mL ampicillin. Turbid broth, indicating an increase in cell density, was observed at the conclusion of the sampling period for the run containing E. coli but not in the broth control. Following in vitro VOC sampling and analysis by GC×GC-TOFMS, chromatographic peaks were aligned in ChromaTOF®, yielding 196 unique features after the removal of known contaminants and compounds observed in the instrument blanks. For statistical analysis, compounds not detected in at least 80% of all samples were removed to focus on the reproducible aspects of the E. coli volatilome. VOCs specific to the E. coli or broth volatilome were also retained, resulting in a total of 108 features. Results show the Biodome has sufficient sensitivity to identify low abundance bacterial-derived VOCs, even when initially present in the culture media (FIG. 7A to FIG. 7D).

Using the reporting standards [Sumner, L. W. et al., Metabolomics 3, 211-221 (2007)] mentioned previously, filtered VOCs were assigned a name and functional group where possible (FIG. 7A). Higher confidence VOCs with an ID level<4 are provided in Table 2. VOCs uniquely observed in the broth may suggest consumption by the bacteria during log and stationary phases of growth, although additional work is needed to validate endogenous origin (FIG. 7A). Using the filtered bacterial and broth volatilomes, PCA and observe highly separated groups were performed when considering the first two components (FIG. 7B). Interestingly, spatial arrangement shows that the E. coli volatilome produced during the first 48 hours of growth is relatively consistent and suggests the third day of sampling may capture the transition to the death phase (FIG. 7B). After adjusting for multiple hypothesis testing using the Benjamini-Hochberg procedure [Benjamini, Y. et al., Journal of the Royal statistical society: series B (Methodological) 57, 289-300 (1995)], 16 VOCs were found to be significantly elevated in the condition containing ampicillin-resistant E. coli (FIG. 7C, FIG. 7D; Table 2).

FIG. 7A through FIG. 7D show the application of the Biodome tool to characterize the E. coli volatilome across the log and stationary phases of growth. FIG. 7A shows a Venn diagram showing the number of unique and common VOCs detected in the bacterial and broth conditions. Pie charts show the breakdown of VOC functional groups specific to each group. FIG. 7B shows a PCA analysis using high confidence VOCs presented in Table 2 showing the E. coli volatilome is distinct from the broth control. FIG. 7C shows line plots for four high confidence VOCs showing log₁₀ transformed abundance across three days of sampling. Day one represents the cumulative volatilome between 0-24 hours of growth, day 2 between 24-48 hours, and the final day capturing 48-72 hours. FIG. 7D shows boxplots for the same four VOCs showing expression of these metabolites are significantly elevated in the E. coli experimental condition, after correcting for multiple hypothesis testing using the Benjamini-Hochberg p-value adjustment procedure [Benjamini, Y. et al., Journal of the Royal statistical society: series B (Methodological) 57, 289-300 (1995)].

The majority of the identified volatiles were found in both the broth and bacterial conditions (63.9%), however abundance changes were observed that indicate not all shared VOCs originate from exogenous sources. Lending strength to claims of enhanced sensitivity, indole (FDR p-value=0.010) and decane (FDR p-value=0.058) were elevated in the samples containing E. coli despite also being observed in the liquid broth volatilome, supporting endogenous production (FIG. 5C, FIG. 5D). Furthermore, noise due to plastics (xylenes and styrene) accounted for less than 1% of the total chromatographic signal in each replicate, emphasizing that the Biodome effectively addresses one of the major existing limitations with existing approaches. Finally, of the 15 VOCs found uniquely in the E. coli volatilome, roughly 75% (11/15) had a median signal-to-noise ratio<150:1, across the log and stationary phases of growth, demonstrating the analyte sensitivity advantages of the tool for untargeted volatile metabolomics.

Many of the VOCs observed in the E. coli volatilome have been previously linked to this microorganism, however the production of low abundance VOCs 1-phenyl-2-butanone, 1-propanol, 2-methyl-3-hexanone, benzyl alcohol, cyclobutane, and methacrolein during the log and stationary phases of growth were first reported (FIG. 5C, FIG. 5D; Table 2). All newly reported VOCs had a median signal-to-noise ratio<150:1. VOCs distinguishing the log phase from the stationary phase of growth were briefly considered and note distinct production of 1-butanol, a dimethylpyrazine isomer eluting after 2,5-dimethylpyrazine, and an unknown compound with average first- and second-dimension retention times of 940 seconds and 1.05 seconds respectively, corresponding to a retention index of 841.

TABLE 3
E. Coli and LB Broth VOCs with an ID level <4

| | | FDR | Avg | Avg | | |
|---|---|---|---|---|---|---|
| | | adjusted | 1tR | 2tR | | ID |
| VOC | Chemical Class | p-value | (s) | (s) | RI | Level |
| Acetone | Ketone | 0.5939 | 639 | 0.67 | 652* | 2 |
| Methacrolein | Aldehyde | 0.042 | 702 | 0.69 | 687* | 2 |
| 1-Propanol | Alcohol | 0.0278 | 709 | 1.02 | 691* | 2 |
| Cyclobutane | Hydrocarbon | 0.0769 | 799 | 1.06 | 758* | 3 |
| 2,3,5-Trimethylhexane | Hydrocarbon | 0.6285 | 994 | 0.65 | 865 | 3 |
| 2,3-Hexanedione | Ketone | 0.437 | 1003 | 0.85 | 869 | 2 |
| 2,3,4-Trimethylhexane | Hydrocarbon | 0.6684 | 1028 | 0.66 | 880 | 3 |
| Nonane | Hydrocarbon | 0.6285 | 1084 | 0.65 | 906 | 2 |
| 5-Methyl-2-hexanone | Ketone | 0.672 | 1141 | 0.88 | 932 | 2 |
| 2-Methyl-3-hexanone | Ketone | 0.0118 | 1156 | 0.82 | 939 | 3 |
| 2,5-Dimethylpyrazine | Heteroaromatic | 0.65 | 1199 | 1.11 | 959 | 2 |
| Cyclohexanone | Ketone | 0.856 | 1232 | 1.05 | 974 | 2 |
| Decane | Hydrocarbon | 0.0584 | 1326 | 0.67 | 1019 | 2 |
| Dimethyl trisulfide | Other | 0.2917 | 1358 | 1.09 | 1034 | 2 |
| Trimethylpyrazine | Heteroaromatic | 0.2161 | 1369 | 1.06 | 1040 | 2 |
| 1,2,3-Trimethylbenzene | Aromatic Hydrocarbon | 0.856 | 1370 | 0.9 | 1040 | 2 |
| 2-Ethyl-5-methylpyrazine | Heteroaromatic | 0.5463 | 1377 | 1.03 | 1044 | 2 |
| 2,2,7,7-Tetramethyloctane | Hydrocarbon | 0.566 | 1392 | 0.66 | 1051 | 3 |
| 2,2,4,4-Tetramethyloctane | Hydrocarbon | 0.7921 | 1395 | 0.67 | 1053 | 3 |
| 2,3,5,8-Tetramethyldecane | Hydrocarbon | 0.7679 | 1438 | 0.67 | 1073 | 3 |
| 2-Acetylthiazole | Azole | 0.1401 | 1462 | 1.53 | 1085 | 2 |
| 2-Methyl-3-isopropylpyrazine | Heteroaromatic | 0.5613 | 1482 | 0.97 | 1095 | 2 |
| Benzyl alcohol | Functionalized Benzene | 0.0133 | 1510 | 0.35 | 1109 | 2 |
| 3-Ethyl-2,5-dimethylpyrazine | Heteroaromatic | 0.3556 | 1526 | 0.99 | 1118 | 2 |
| 2-Hydroxybenzaldehyde | Other | 0.5586 | 1534 | 1.54 | 1122 | 2 |
| 1-Octanol | Alcohol | 0.856 | 1536 | 1.11 | 1123 | 2 |
| 2-Nonanone | Ketone | 0.0094 | 1581 | 0.86 | 1146 | 2 |
| Benzoic acid, methyl ester | Functionalized Benzene | 0.938 | 1605 | 1.23 | 1159 | 2 |
| 2-Methylundecane | Hydrocarbon | 0.5178 | 1628 | 0.7 | 1171 | 2 |
| 3,5-Diethyl-2-methylpyrazine | Heteroaromatic | 0.437 | 1666 | 0.94 | 1191 | 2 |
| (E)-2-Nonenal | Aldehyde | 0.5242 | 1729 | 0.99 | 1225 | 2 |
| 1-Phenyl-1-propanone | Functionalized Benzene | 0.5178 | 1760 | 1.24 | 1242 | 2 |
| Benzyl nitrile | Functionalized Benzene | 0.5713 | 1766 | 1.76 | 1246 | 2 |
| 3-Butyl-2,5-dimethylpyrazine | Heteroaromatic | 0.5178 | 1848 | 0.94 | 1291 | 2 |
| 1-Phenyl-2-butanone | Functionalized Benzene | 0.0088 | 1881 | 1.21 | 1310 | 2 |
| Indole | Heteroaromatic | 0.0097 | 2100 | 1.2 | 1442 | 2 |
| 3-hydroxy-2,2,4-trimethylpentyl | Other | 0.1074 | 2129 | 1 | 1460 | 3 |
| isobutyrate | | | | | | |

Shown in Table 3, the compound name is presented first, followed by the functional group class. Bolded names indicate that the VOC was not detected in the broth controls. The adjusted p value was calculated using a two-tailed student's t-test, comparing the LB broth and Escherichia coli volatilomes post-filtering. The first (¹t_{R}) and second (²t_{R}) dimension retention times are included, with the retention index calculated using a C8-C20 alkane standard mix. Finally, the VOC naming confidence level is reported according to previously established standards.

In the disclosed example, a glass culture vessel was first worked on to facilitate the in vitro analysis of biological volatilomes. The functionality of this tool by integrating computational modeling and fluorescent imaging to demonstrate desirable sampling and growth conditions was validated. The origin of VOCs from the flow system itself were extensively characterized as was the culture media and the human-derived SK-OV-3 ovarian adenocarcinoma cells. In doing so, at least two compounds not previously observed in vitro but reported in vivo were identified. To further verify endogenous origin, and demonstrate the variety of methods supported by the tool, ¹³C₆-D-glucose-derived VOCs was globally labeled and 2-Decen-1-ol with mass spectral data supporting ¹³C incorporation was identified. With a similar aim, lipid metabolism leveraging RNAi-mediated knockdown of CPT2 were inhibited and four additional volatile metabolites that show a decrease in abundance consistent with the transient recovery of CPT2 expression across the 4 days of analysis were found. Finally, to broaden the scope of use for in vitro volatile metabolomics, DH5α Escherichia coli volatilome across the log and stationary phases of growth (0-72 hours) was characterized and six low abundance VOCs not previously reported were identified.

Considering the newly reported SK-OV-3 cell volatile metabolites [Filipiak, W. et al., Current medicinal chemistry 23, 2112-2131 (2016)], 2-ethyl-1-hexene (3-methyleneheptane) has not been previously detected in vitro, however multiple studies report observing this VOC in fecal samples [Garner, C. E. et al., The FASEB Journal 21, 1675-1688 (2007)] and in the breath of both healthy individuals [de Lacy Costello, B. et al., Journal of breath research 8, 014001 (2014)] and in patients following hyperbaric oxygen therapy [de Jong F. J. et al., Metabolites 13, 316 (2023); Wingelaar, T. T. et al., Frontiers in Physiology 10, 10 (2019)], potentially linking this VOC to cellular respiration. Interestingly, following ¹³C-glucose supplementation, a variable but consistent shift in the parent ion (m/z 112) corresponding to 1-6 ¹³C molecules incorporated (113-118 amu) was observed, however the original parent fragment is inconsistently observed limiting interpretation. Further to the contrary, 2-ethyl-1-hexene has been reported as an atmospheric VOC [Xu, X. et al., Atmospheric Chemistry and Physics 3, 665-682 (2003)], indicating exogenous sources cannot be ruled out entirely, although the lack of observation in media and flow system controls supports cellular origin. To the best of the authors' knowledge, very little has been reported on 2-methyl-2-dodecanol as a mammalian metabolite [Filipiak, W. et al., Current medicinal chemistry 23, 2112-2131 (2016); de Lacy Costello, B. et al., Journal of breath research 8, 014001 (2014)]. However, 1-dodecanol, similar in structure, has been shown to act as a pheromone in non-mammalian organisms [Tian, Z. et al., Pest Management Science 76, 3667-3675 (2020), Farine, J. P. et al., Journal of chemical ecology 20, 2291-2306 (1994)] potentially indicating a relationship to the ovarian adenocarcinoma cell line utilized in this study. Tridecanoic acid methyl ester has not been previously reported in mammalian cell culture in vitro, however both the free fatty acid (tridecanoic acid) and esterified form have been detected from healthy human skin [de Lacy Costello, B. et al., Journal of breath research 8, 014001 (2014); Gallagher, M. et al., British Journal of Dermatology 159, 780-791 (2008)]. Similarly, dodecyl (lauryl) acrylate has not been reported in vitro but has been detected from human skin by direct PDMS contact [Wooding, M. et al., Analytical and Bioanalytical Chemistry 412, 5759-5777 (2020)]. Dodecyl acrylate is known to be produced by the condensation of 1-dodecanol (HMBD0011626) with acrylic acid (HMBD0031647), both of which are known human metabolites [Wishart, D. S. et al., Nucleic acids research 50, D622-D631 (2022)], potentially indicating its role as a secondary marker for these metabolites. To the contrary, 1-dodecanol was one of the more abundant contaminants present in the flow system, suggesting observation may strictly be an indicator of endogenous acrylic acid, although additional work is needed to verify this origin. In general, the results demonstrate that the Biodome with imaging tray effectively enhances total signal recovery for in vitro mammalian volatilomics and allows reproducible detection of low-abundance, intracellular VOCs.

Mammalian cell VOCs worth further consideration include 1,2,3-trimethylbenzene (hemimellitene), 1-butanol, 2,4,6-trimethylheptane, 2-methylhexane, and 3-methylbutanal (isovaleraldehyde). 1,2,3-trimethylbenzene has been reported in vitro in growth media controls and significantly elevated by in situ SPME and gas subsampling in A549 lung carcinoma cells [Schallschmidt, K. et al., Journal of chromatography B 1006, 158-166 (2015)], suggesting endogenous origin. Here, the results show additional evidence supporting cellular origin and further find agreement in published literature with 1,2,3-trimethylbenzene reported in healthy human feces and breath56. Furthermore, 1,2,3-trimethylbenzne has been linked to recurrent wheezing in children and gastrointestinal disease in adults, suggesting a role in human disease [Garner, C. E. et al., The FASEB Journal 21, 1675-1688 (2007); van de Kant K. D. et al., European Respiratory Journal 41, 183-188 (2013)]. 1-Butanol has been previously reported in vitro in A549 lung carcinoma cells [Hanai, Y. et al., Cancer cell international 12, 1-3 (2012)], although some limitations to biological interpretation exist as covered by Schallschmidt et al. [Schallschmidt, K. et al., Journal of chromatography B 1006, 158-166 (2015)]. The results provide additional evidence for endogenous origin and the findings are further supported by the detection of 1-butanol in most human biofluids56 with increases reported in the urine and serum of diabetic patients [Liebich, H. M. et al., Journal of Chromatography A 239, 343-349 (1982)], suggesting a role in human disease. To best knowledge, 2,4,6-trimethylheptane has not been previously observed in the in vitro mammalian volatilome. However, this VOC has been reported in the breath of healthy individuals [Xu, X. et al., Atmospheric Chemistry and Physics 3, 665-682 (2003)], lending support to the findings. 2-Methylhexane has been previously reported in vitro [Schallschmidt, K. et al., Journal of chromatography B 1006, 158-166 (2015)], with decreases in abundance relative to the media control, suggesting exogenous origin and/or bioconversion by the cells. Here evidence to the contrary was found—given a single observation across media and flow system controls and consistent detection in the cellular volatilome. In agreement with the findings, 2-methylhexane has been reported in the breath of healthy individuals [de Lacy Costello, B. et al., Journal of breath research 8, 014001 (2014)] and in patients with lung cancer, acting as a discriminatory marker for the disease [Phillips, M. et al., Chest 123, 2115-2123 (2003); Kischkel, S. et al., Clinica Chimica Acta 411, 1637-1644 (2010)], suggesting a role as a human metabolite. Interestingly, 3-methylbutanal has been previously observed in vitro from NCI-H2087 lung adenocarcinoma cells using an early dynamic headspace sampling approach and large bioreactor system developed by Sponring et al. [Sponring, A. et al., Anticancer research 29, 419-426 (2009)]. Results from Sponring et al. indicate exogenous presence in the culture media/bioreactor, while the findings support intracellular origin given the lack of observation in controls. Sponring et al. hypothesize differences in culture conditions, nutrient availability, and the optimization of headspace-to-volume ratio in the Biodome may partially explain the apparent differences.

The results from isotopic labeling with ¹³C₆-D-glucose confirm 2-Decen-1-ol as an endogenous VOC derived from glycolysis. 2-Decen-1-ol is a fatty alcohol that falls under a class of molecules synthesized from elongation of an acetyl-CoA substrate. In line with established lipid biosynthetic pathways, the mass spectral data supports the incorporation of ¹³C-labeled acetyl-CoA with carbon isotopes near the alcohol functional group. Subsequent metabolic events leading to the production of 2-Decen-1-ol include chain elongation of acetyl-CoA and, eventually, fatty alcohol formation via fatty acyl-CoA reductase. Complex labeling patterns observed for 2-Decen-lol arise from variable degrees of ¹³C incorporation in each of these biosynthetic pathways. Extensively labeled molecules corresponding to larger mass shifts depict higher degrees of ¹³C incorporation. Small mass shifts indicating a lower degree of ¹³C incorporation could potentially arise from the elongation of unlabeled fatty acids and precursors that also feed into lipid metabolism, and/or a lower degree of ¹³C-labeled acetyl-CoA added during elongation. Fluctuating degrees of ¹³C-labeled fatty acids were also observed by Kamphorst, et al., who used both ¹³C-labeled glucose and glutamine to study the kinetics of fatty acid metabolism [Kamphorst, J. J. et al., Analytical chemistry 83, 9114-9122 (2011)]. 2-Decen-1-ol and other fatty acid metabolites are energy-rich compounds that can be utilized for cellular signaling, membrane formation, and energy production [Guertin, D. A. et al., Nature Reviews Cancer 23, 156-172 (2023); Snaebjornsson, M. T., Cell metabolism 31, 62-76 (2020)]. Furthermore, a considerable number of studies indicate that these biological processes are altered in cancer cells to promote survival and tumorigenesis [Guertin, D. A., Wellen, K. E., Acetyl-CoA metabolism in cancer. Nature Reviews Cancer 23, 156-172 (2023); Snaebjornsson, M. T. et al., Cell metabolism 31, 62-76 (2020); Wei, X. et al., Frontiers in Oncology 11, 642229 (2021)]. Notably, upregulated fatty acid metabolism plays a key role in ovarian cancer cell survival, and the expression of genes encoding acetyl-CoA metabolic enzymes is associated with increased aggressiveness and poor prognosis [Snaebjornsson, M. T. et al., Cell metabolism 31, 62-76 (2020); Wei, X. et al., Frontiers in Oncology 11, 642229 (2021)]. Taken together, these findings highlight the ability of the Biodome tool to effectively capture VOCs of key biological processes in vitro, using an untargeted approach to identify metabolites associated with disease.

Like 2-Decen-1-ol, many VOCs detected from mammalian culture in vitro often feature a relatively long hydrocarbon backbone and are generally thought to originate from fatty acid metabolism. To more generally evaluate the role of fatty acid metabolism in the production of VOCs in vitro, mitochondrial lipid transporter protein carnitine palmitoyltransferase 2 (encoded by CPT2) using RNA interference was elected to transiently inhibit its expression. 2-Ethyl-1-hexene, dodecyl acrylate, tridecanoic acid methyl ester, and an unidentified alkene were strictly observed in cellular volatilomes and demonstrated abundance decreases consistent with the knockdown of CPT2 across 4 days. As would be expected, all compounds feature a hydrocarbon backbone. 2-Ethyl-1-hexene and the unidentified alkene include a double bond which may indicate origin from an unsaturated fatty acid, while dodecyl acrylate and tridecanoic acid methyl ester are likely derived from saturated fatty acids. The work of the disclosed example represents the first application of RNAi to assess pathway origin of VOCs in mammalian cells, however similar success has been recently demonstrated in plants [Adebesin, F. et al., Science 356, 1386-1388 (2017); Liao, P. et al., Nature Communications 14, 330 (2023)]. Although a specific enzymatic step cannot be assigned to their production, the findings strengthen claims for cellular origin and provide a methodological platform for the secondary verification of VOC producing pathways in mammalian volatilomes.

In the case of the E. coli volatilome, the significant increase in indole, relative to the broth control, is an indication of the advantages of the Biodome tool for in vitro volatile metabolomics. Conversion of tryptophan to indole by tryptophanase is a well characterized process in E. coli [Newton, W. A. et al., Proceedings of the National Academy of Sciences 51, 382-389 (1964); Newton, W. A. et al., Journal of Biological Chemistry 240, 1211-1218 (1965)]. Tryptophanase catalyzes the production of pyruvate in conditions of excess tryptophan, as expected in starter broth cultures [Li, G., Young, K. D., Microbiology 159, 402-410 (2013)], generating indole as a byproduct [Newton, W. A. et al., Proceedings of the National Academy of Sciences 51, 382-389 (1964); Newton, W. A. et al., Journal of Biological Chemistry 240, 1211-1218 (1965)]. Here, indole was found to be the most abundant peak across the log and stationary phases of growth (0-72 hours) in the E. coli volatilome, although one recent study has shown bacterial transformation to induce ampicillin resistance may act as a confounding factor [Dixon, B. et al., Journal of Applied Microbiology 133, 2445-2456 (2022)]. Generally, the results agree with many previous studies, which demonstrate indole is a biomarker for E. coli, acting as an extracellular signaling molecule with import and export complexes, and serving to regulate biofilm formation, cell division, and gene expression [Bos, L. D. et al., PLoS pathogens 9, e1003311 (2013); Wang, D. et al., Journal of Bacteriology 183, 4210-4216 (2001); Di Martino, P., et al., Canadian journal of microbiology 49, 443-449 (2003); Chimerel, C. et al., Biochimica et Biophysica Acta (BBA)-Biomembranes 1818, 1590-1594 (2012); Hirakawa, H. et al., Molecular microbiology 55, 1113-1126 (2005)]. Worth discussing, indole was detected with high confidence in the broth controls and had similarly broad peaks. Therefore, to ensure consistency, peaks within the first-dimension retention time window of 2092-2110 seconds were summed in all chromatograms, likely explaining the high reported signal in broth controls despite clear differences in abundance. Demonstrating similar agreement, recent studies also report 2-nonanone in the E. coli volatilome, including the DH5a strain similar to in the disclosed example [Sousa, M. et al., Food Control 146, 109513 (2023); Almeida, O. A. et al., Frontiers in Plant Science 13, 1056082 (2023)]. Further, biosynthesis of 2-nonanone is known to be naturally derived from the decarboxylation of 10 carbon β-keto acids [Schulz, S., Dickschat, J. S., Natural product reports 24, 814-842 (2007)] and has been shown to regulate gene expression associated with quorum sensing [Plyuta, V. A. et al., Molecular Genetics, Microbiology and Virology 29, 167-171 (2014)]. Taken together, the findings demonstrate that volatile profiles acquired using the Biodome tool and imaging tray accurately reflect active metabolic processes in E. coli and agree with previous studies in regard to the abundance of these VOCs relative to the remainder of analytes comprising the volatilome.

While the well characterized E. coli VOCs indole and 2-nonanone was detected, Observations of 1-phenyl-2-butanone, 2-methyl-3-hexanone, 1-propanol, benzyl alcohol, cyclobutane, and methacrolein in the DH5α E. coli volatilome but not in the LB broth control were first reported. Interestingly, while 1-phenyl-2-butanone has not been previously observed in vitro, the structural analog 3-hydroxy-1-phenyl-2-butanone has been observed in other microorganisms [Asakawa, T. et al., Biochimica et Biophysica Acta (BBA)-General Subjects 170, 375-391 (1968); Guo, Z. et al., Tetrahedron: Asymmetry 10, 4667-4675 (1999)] with a natural biosynthesis pathway reported via the condensation reaction of phenylpyruvic acid with acetaldehyde, catalyzed by phenylpyruvate decarboxylase [Ward, O. P. et al., Current opinion in biotechnology 11, 520-526 (2000)]. Alternatively, oxidation of trans-1-phenyl-1-butene by rat cytochrome P-450 has been shown to produce 1-phenyl-2-butanone. Given previous works, the Ward, O. P. et al. proposed further catabolism of 3-hydroxy-1-phenyl-2-butanone to give 1-phenyl-2-butanone and/or oxidation by cytochromes—both suggesting intracellular origin. To best knowledge, 2-methyl-3-hexanone has not been previously detected in the E. coli volatilome, although the reaction of 3-oxoacids with H+ to produce methyl ketones and CO2 is known to occur intracellularly and spontaneously [Keseler, I. M. et al., Nucleic acids research 45, D543-D550 (2017)], supporting endogenous origin.

Further highlighting analyte recovery advantages using the Biodome, the results support the intracellular production of 1-propanol and benzyl alcohol. Most studies work to metabolically engineer E. coli to produce appreciable quantities of 1-propanol [Shen, C. R. et al., Metabolic engineering 10, 312-320 (2008); Choi, Y. J. et al., Metabolic engineering 14, 477-486 (2012)] and benzyl alcohol [Pugh, S. et al., Metabolic Engineering Communications 2, 39-45 (2015)]. The former is known to be produced intracellularly as a product of β-D-glucuronide and D-glucuronate degradation [Keseler, I. M. et al., Nucleic acids research 45, D543-D550 (2017)], isoleucine biosynthesis via 2-keto acid decarboxylase and alcohol dehydrogenase [Atsumi, S. et la., Nature 451, 86-89 (2008)], and in association with sleeping beauty mutase (Sbm) operon in wild-type E. coli, although expression is minimal [Haller, T. et al., Biochemistry 39, 4622-4629 (2000); Srirangan, K. et al., Applied microbiology and biotechnology 98, 9499-9515 (2014)]. In agreement with these works, the results support intracellular production given the low abundance of 1-propanol, and further suggest detectable abundance has remained beyond the capabilities of traditional equilibrium-based sampling methodologies. Similarly, benzyl alcohol has been shown to be produced endogenously from benzaldehyde and multiple native alcohol dehydrogenases and aldo-keto reductases [Pugh, S. et al., Metabolic Engineering Communications 2, 39-45 (2015); Sulzenbacher, G. et al., Journal of molecular biology 342, 489-502 (2004)]. Further, benzyl alcohol has been shown to induce heat shock proteins in E. coli [De Marco, A. et al., Cell stress & chaperones 10, 329 (2005)], suggesting a functional role for this volatile metabolite.

Cyclobutane has not been previously reported as a VOC originating from E. coli. While the natural biosynthetic routes are not well characterized [Tang, B. et al., Organic letters 21, 1243-1247 (2019); Hong, Y. J. et al., Chemical Society Reviews 43, 5042-5050 (2014)], cyclobutane is known to be produced intracellularly during the dimerization of pyrimidine nucleotides as a consequence of UV-induced DNA damage [Setlow, R. B. et al., Science 153, 379-386 (1966)], yet repair mechanisms do not release cyclobutene [Liu, Z. et al., Proceedings of the National Academy of Sciences 108, 14831-14836 (2011)], potentially indicating exogenous origin. To the contrary, cyclobutane has been observed in the feces and breath of healthy humans [de Lacy Costello, B. et al., Journal of breath research 8, 014001 (2014)], suggesting a role as a microbial metabolite. Additional support for intracellular origin given cyclobutane was observed with a median signal-to-noise ratio<100:1 was found. Finally, while little is known about the intracellular production of methacrolein, studies have shown that the non-mevalonate pathway for isoprenoid biosynthesis is active in E. coli [Takahashi, S., et al., Proceedings of the National Academy of Sciences 95, 9879-9884 (1998); Charon, L. et al., Biochemical Journal 346, 737-742 (2000)] and that isoprene oxidation produces methacrolein [Warneke, C. et al., Journal of Atmospheric Chemistry 38, 167-185 (2001)], with evidence supporting direct biological emission from plants [Jardine, K. J. et al., Global Change Biology 18, 973-984 (2012)] and other microorganisms [Trefz, P. et al., PloS one 8, e76868 (2013)]. However, cellular origin cannot be definitively established, with evidence supporting an exogenous source including the absence of isoprene. Generally, the consideration of the E. coli volatilome emphasizes the analyte recovery advantages of the Biodome tool, expanding depth of coverage and enabling study of previously uncharacterized volatile metabolites.

In brief summary, the findings highlight the advantages of the Biodome tool for in vitro volatile metabolomic analysis, which include (i) dynamic headspace methodology to enhance total signal recovery allowing detection of lower abundance VOCs, (ii) application of borosilicate glass to allow sterilization and reuse, limit exogenous signal due to plastics, and support dual imaging function, and (iii) improved reproducibility while operating in the laminar flow range. The non-destructive nature of collection allows for analysis of VOCs across time and in response to perturbation, as demonstrated by the isotopic labeling and RNA interference applications. The modular system design facilitates adaptation to microbiological analysis, with notable usefulness for the study of anaerobes and extremophiles, and compatibility through the analysis of the DH5α Escherichia coli volatilome across the log and stationary phases of growth was demonstrated.

Biodome Development and Fabrication: A CAD model of the Biodome culture vessel, with the TDT adapter included, was designed in SOLIDWORKS® 2019. Flow characteristics were then modeled in the CAD part using ANSYS Fluent® 2019 R3. For meshing, the target skewness was adjusted to 0.65, smoothing set to high, a mesh metric set to “Orthogonal Quality”, and default for all other parameters. The inlet and outlet mesh were then refined using a value of 2. Boundary conditions were then set, using inlet velocities ranging from 1-50 mL/min and the pressure outlet condition. The operating pressure was assumed to be 1 atm, the fluid set to air, and acceleration due to gravity was included in the model. The residual threshold was adjusted to LE-5. Solution parameters were as follows: Pressure-Velocity Coupling Scheme set to SIMPLE, Spatial Discretization Gradient set to Least Squares Cell Based, Pressure set to Second Order, and Momentum set to Second Order Upwind. Solution results were visualized using ANSYS CFD Post GUI.

Fabrication of the glass culture vessel leverages specialized trade techniques, but in brief begins with rotation of Pyrex tubing using a glassblowing lathe. A natural gas or oxygen flame was used to pull the tube down into a flat bottom. The inlet and outlet were then added to onto the flat bottom. Using a custom holder to retain that piece in the lathe, another flat bottom was pulled to create the internal chamber. Additional attention was given to the curvature of the bottom and top to ensure the flattest surface possible for imaging applications.

To assemble the TDT adapter, Nalgene tubing was inserted into the 7.9375 mm nut head (with moderate force) and the other end was inserted into the rubber stopper. A metal ferrule and tubing insert was attached to the free end and the nut-ferrule assembly was then connected to the male-to-male adapter. A ferrule was then added to the 6.35 mm stainless steel nut and screwed firmly into the other end of the male-to-male adapter. The opening of the 6.35 mm stainless-steel nut served as the interface for TDT sampling. An airtight seal was ensured by wrapping 7-8 cm of oxygen-compatible PTFE tape around the beveled end of the TDT tube before inserting it into the TDT interface. A visible decrease in the flow rate (>2 mL/min) was always observed when the TDT was inserted into the adapter and the regulator was readjusted to achieve the original flow rate after roughly 5-10 minutes of equilibration. To adapt the system for SPME sampling methodology, a 1 mL disposable syringe body (Electron Microscopy Sciences, Hatfield, PA), with the plunger removed, in line with the 6.35 mm (¼ inch) nut (replacing the TDT) and completely sealing the interface with oxygen-compatible PTFE. The SPME fiber then rests naturally at the narrow end of the syringe body—without contacting the sides of the syringe. Other SPME adapters were considered, including the use of a GC SPME glass liner insert, however the added volume of the syringe was found to help with the condensation of water during sampling durations>24-48 hours (depending on flow rate).

Cell culture: SK-OV-3 ovarian adenocarcinoma cells (human origin; HTB-77) were obtained from ATCC. Cells were maintained in RPMI 1640 (Sigma-Aldrich, St. Louis, MO, USA) supplemented with 10% FBS (ATCC) and 1% penicillin (100 I.U./mL; ATCC)/streptomycin (100 μg/mL; ATCC). Cells were maintained in a humidified incubator at 37° C. and 5% CO2. Cultures were grown in standard T-75 flasks and passaged at 70-85% confluency.

To seed the Biodome for VOC analysis, approximately 500,000 cells—estimated using a hematocytometer—were seeded in the Biodome. Cells were allowed to adhere from within a standard incubator for 24 hours, and the culture media was replaced immediately before the start of analysis. Vented caps from standard T-25 flasks (VWR) were allowed to rest on top of the inlet and outlet of the Biodome—maintaining an enclosed environment and CO₂ exchange with the media. The total media volume in the Biodome was maintained at 5 mL for all experiments. To limit the background signal from the culture media, dialyzed fetal bovine serum (FBS) was used in place of traditional FBS, ensuring small molecule (<10,000 Da) concentration was reduced.

Finally, to improve the consistency of heating and reduce condensation, the Biodome was completely submerged within a bead bath maintained at 37° C. Gas flow was set 24 hours before the start of experimentation to ensure an equilibrium was reached within the two-stage regulator.

Bacterial Culture: DH5α E. coli were transformed with plasmid containing the ampicillin resistance gene (pUC19, Addgene Plasmid #50005) and streaked on LB agar plates spiked with 100 g/mL ampicillin. Isolated colonies were inoculated in 100 g/mL ampicillin spiked LB broth and grown for 24 hours. OD₆₀₀ measurements were taken and used to dilute the bacterial culture such that the initial OD₆₀₀ seeding concentration was approximately 0.1 (true value=0.099) at the start of VOC collection. Bacteria were seeded into the Biodome containing a total of 5 mL of LB broth spiked with 100 g/mL ampicillin and TDU tubes were replaced every 24 hours. The carrier gas composition was 95% air/5% CO₂, to allow compatibility with cellular analysis, and a flow rate of 18 mL/min was maintained for the duration of sampling. Gas flow was set 24 hours before the start of experimentation to ensure an equilibrium was reached within the two-stage regulator.

Fluorescent Imaging: Prior to the start of the live/dead assay, seeded cells were allowed to adhere for 24 hours in a humidified incubator at 37° C. and 5% CO₂ and culture media was replaced immediately before introduction to the flow system. The Biodome was then incubated in the flow system for durations spanning 0 and 4 days. Following growth in the flow system, live/dead fluorescent probes (Blue/Green ReadyProbes, Invitrogen) were added to the Biodome according to the manufacturer guidelines. In brief, 2 drops of the ready-to-use stain were added per millimeter, totaling 8 drops. The biodome was then placed in a humidified incubator at 37° C. and 5% CO₂ for 30 minutes. After 30 minutes, the Biodome was imaged using Eclipse Ts2R microscope (Nikon, Minato City, Tokyo, Japan) with a Coolsnap Dyno CCD (Photometrics, Tucson, Arizona, USA) and 10× objective. A FITC filter (EX: 482 nm, EM: 536 nm; Nikon) was used to excite the live stain (Hoechst 33342) while a DAPI filter (EX: 390 nm, EM: 475 nm; Nikon) was used to visualize the dead stain. Fluorescent image processing and cell counting was performed using ImageJ.

To evaluate cell morphology, actin fluorescence staining was performed in live cells using CellMask™ Orange Actin Tracking Stain (Invitrogen). Cells were grown out in the specified incubation system for approximately 3 days until the confluency had reached 70-80%, 1000× stock solution of the actin tracking stain in DMSO was diluted to 1× in the RPMI1640 culture media described previously. Live/dead fluorescent probes were concurrently added to the staining media at a ratio of 2 drops per milliliter and cells were incubated for 30 minutes in a humidified incubator as previously described. Cells were rinsed twice with a wash buffer comprised of culture media previously equilibrated in a humidified incubator at 5% CO₂ and 37° C. for 30 min. Cells were imaged using a Leica DMi8 (Leica, Wetzlar, Germany) microscope with DFC345 FX monochrome camera (Leica) and Texas Red (EX: 560 nm, EM: 645 nm), DAPI, and FITC filters to visualize the actin, dead cell nuclei, and live cell nuclei, respectively. Images were processed and fluorescent channels overlaid using ImageJ. All fluorescent images were captured using cells at passage<10 using a 10× objective.

Stable isotope labeling of metabolites in cell culture: RPMI 1640 base medium without sodium bicarbonate and glucose was used (Sigma Aldrich), supplemented with 2 g/L 13C6-D-glucose (Cambridge Isotope Laboratories) and sodium bicarbonate (Sigma Aldrich). The final media contained 10% dialyzed FBS and 1% penicillin/streptomycin. To ensure equivalency when assessing isotopically labeled volatilomes, SK-OV-3 cells were passaged 20 times in their respective medias. Labeled and unlabeled cells were independently seeded into the glass culture vessel and allowed to grow out for 24 hours before starting sampling. Prior to connecting the Biodome to the flow system, culture media was replaced to ensure previous accumulation of VOCs did not influence results.

¹³C Peak Alignment: ¹³C₆-D-glucose was chosen as the labeling method because it allows peak alignment using retention times, given ¹³C and ¹²C isomers elute from the column at the same time [Berg, T. et al., Journal of Chromatography A 1344, 83-90 (2014); Kempa, S. et al., Journal of basic microbiology 49, 82-91 (2009)]. Chromatograms were first aligned within each experimental condition (i.e., labeled, and unlabeled) using ChromaTOF Statistical Compare package. Then, to assist with the identification of ¹³C-labeled VOCs, custom R code was developed (https://github.com/BSmithLab/Biodome) to facilitate alignment of unlabeled and labeled peaks by providing external control over the retention time variability. For each given peak in the unlabeled condition, all ¹³C-labeled peaks that fell within the allowed retention time window were identified, regardless of their underlying mass spectrum. This approach was deemed necessary to ensure chromatographic alignment between labeled and unlabeled conditions did not apply spectral deconvolution, which influences the resulting mass spectrum assigned to each peak. Mean retention times were determined for all ¹³C labeled and unlabeled peaks, and the first-dimension retention time threshold was set at 10 seconds while the second-dimension was set to 0.07 seconds. Labeled and unlabeled mass spectrums were then manually compared.

RNA inhibition: Pooled siRNA oligonucleotides were purchased from Dharmacon (Lafayette, CO, USA) targeting CPT2 [siGENOME SMARTpool Human CPT2; 5′-GGCAGAAGCUGAUGAGUAG-3′ (SEQ ID NO:1), 5′-UGGCAUACCUGACCAGUGA-3′ (SEQ ID NO:2), 5′-GGAAAGUGGACUCGGCAGU-3′ (SEQ ID NO:3), 5′-CAAGAGACUCAUACGCUUU-3′(SEQ ID NO:4)] and a non-silencing (sham) control [siGENOME Non-Targeting siRNA Pool #1; 5′-UAGCGACUAAACACAUCAA-3′ (SEQ ID NO:5), 5′-UAAGGCUAUGAAGAGAUAC-3′ (SEQ ID NO:6), 5′-AUGUAUUGGCCUGUAUUAG-3′ (SEQ ID NO:7), 5′-AUGAACGUGAAUUGCUCAA-3′ (SEQ ID NO:8)]. SK-OV-3 cells were grown in a 6-well format and transfected at 80% confluency with complexes consisting of 50 nM pooled RNAi probes and 2 g/mL Lipofectamine™ 2000 (Invitrogen) in Opti-MEM® I Reduced Serum Medium (ThermoFisher, Waltham, MA, USA). In the 6-well format, an extra well was allocated for direct seeding into the Biodome for VOC analysis. After 24 hours in a humidified incubator at 37° C. and 5% CO₂, the media was aspirated and the transfected cells were trypsinized. Approximately 500,000 cells were then seeded into the Biodome containing 5 mL of RPMI1640 with 10% dialyzed FBS and 1% Pen/Strep. The cells were allowed to adhere for roughly 1 hour in an incubator before being transferred to the flow system. For the transient RNAi assay, media was replaced after 24 hours, defining the 0-hour time point.

RNA extraction: Following RNAi, cellular RNA was extracted using PureLink® RNA Mini Kit (Life Technologies, Carlsbad, CA, USA) according to manufacturer guidelines. In brief, fresh lysis buffer containing 1% 2-mercaptoethanol (Sigma Aldrich) was used to lyse the cells directly in the 6-well plate. Lysate was homogenized using 10 repeat passes through an 18-gauge needle (BD, Franklin Lakes, NJ). Binding, washing, and elution of purified RNA was performed according to kit specifications. Purity was assessed using absorbance values from a NanoDrop™ One (ThermoFisher Scientific) and all samples were found to have A260/A280 ratios between 1.98-2.06. Samples were stored at −80° C.

RT-qPCR: Purified RNA samples were thawed on ice and subject to reverse transcription using the QuantiTect Reverse Transcription Kit (Qiagen, Germantown, MD, USA). In brief, 100 ng of RNA was used as the starting weight. The genomic DNA elimination and reverse transcription reactions were prepared according to kit specifications. Reverse transcription reaction was allowed to proceed for a total of 15 minutes. Samples were stored for less than two weeks at −20° C. before proceeding to qPCR.

The resulting cDNA was then subject to quantitative PCR to determine expression of CPT2 and housekeeping gene GAPDH. Primers for CPT2 [RefSeq Accession: NM_000098; forward primer 5′-TGGTTTATCTGCCCGTATGC-3′ (SEQ ID NO:9) and reverse primer 5′-TGCTCCAAGTACCATGGC-3′ (SEQ ID NO:10)] and GAPDH [RefSeq Accession: NM_002046; forward primer 5′-ACATCGCTCAGACACCATG-3′ (SEQ ID NO:11) and reverse primer 5′-TGTAGTTGAGGTCAATGAAGGG-3′ (SEQ ID NO:12)] were purchased from IDT (Newark, NJ, USA). The qPCR reaction was prepared in a 96-well format (ThermoFisher Scientific) using iTaq Universal SYBR Green Supermix (Bio-Rad Laboratories, Hercules, CA, USA), according to manufacturer guidelines. In brief, a 20 uL reaction size was selected, using 500 nM of the forward and reverse primer, 1 uL of cDNA, 10 uL of 2× iTaq, and remaining volume balanced with nuclease-free H2O. 96-well plates were transferred to a qTOWER 2.0 instrument and expression quantified using the following cycling conditions: 50° C. for 2 minutes, 95° C. for 5 minutes, followed by 40 cycles of amplification at 95° C. for 15 seconds then 60° C. for 1 minute. Annealing at 72° C. was allowed to proceed for 10 minutes, at which point the dissociation characteristics were assessed by ramping the temperature from 65° C. to 95° C. using a step size of 2° C., equilibration time of 6 seconds, and heating rate of 0.5° C./second. Independent biological replicates were analyzed in technical triplicate and averaged.

GC×GC-TOFMS: Thermal desorption tubes (Carbopack C, Carbopack B, and Carbosieve SIII, see Results) containing retained VOCs were analyzed using comprehensive GC×GC-TOFMS (Pegasus 4D©, LECO Corp. St. Joseph, MI), equipped with an autosampler (Multipurpose Sampler RoboticPro®, Gerstel Inc., Linthicum Heights, MD). TDU tubes were stored for a maximum of 8 days at 4° C. to limit degradation [Harshman, S. W. et al., Journal of breath research 10, 046008 (2016)] and analyzed in order of collection. Volatiles were desorbed into the TDU 2 (Gerstel Inc.) inlet for a total of 4 minutes using variable temperature programming and splitless desorption mode. The TDU 2 inlet was initially held at 50° C. for 30 seconds after which a ramp rate of 700° C./min was applied until the final temperature of 300° C. was achieved. The inlet temperature was held at 300° C. for 3 minutes prior to the start of chromatographic analysis. The transfer temperature was maintained at 310° C. The TDU 2 inlet was paired with a cooled injection system (CIS) to enhance analyte sensitivity. The CIS was initially maintained at −100° C. for the first 30 seconds of the run. The CIS temperature was then ramped at a rate of 12° C./sec until the final temperature of 275° C. was achieved. The CIS was then maintained at 275° C. for 3 minutes.

The instrument was fitted with a two-dimensional column set consisting of a Rxi-624Sil MS (60 m×250 μm×1.4 μm; Restek®, Bellefonte, PA) first dimension column and a Stabilwax (1 m×250 μm×0.5 μm, Restek) second dimension column joined together by a press-fit connection (length×internal diameter×film thickness, respectively). The first-dimension column was set at an initial temperature of 50° C. and held for 2 minutes. The temperature was increased at a rate of 5° C./min until reaching the target temperature of 230° C., where a final hold time of 5 minutes was applied. The secondary oven was maintained at a +5° C. offset relative to the primary oven. A quad-jet modulator was used with a 2 second modulation period and a +15° C. offset relative to the secondary oven. The transfer line was maintained at 250° C. and the ion source at 200° C. for the duration of the run. Helium (UHP, 99.999%) was used as the carrier gas at a flow rate of 2 mL/min. Mass spectra were acquired at 100 Hz over a mass range of 35-300 Da with an ionization energy of −70 eV. Prior to all sample analysis, a PFTBA standard was run to tune the mass spectrometer. Empty vials (blanks) were run at the start of each sample set to monitor the system for contamination. An alkane standard (C8-C20; Sigma Aldrich, St. Louis, MO) was sampled at the conclusion of testing for use in determination of retention indices.

Data Processing: Within individual sample chromatograms, subpeaks in the second dimension were required to meet a signal-to-noise ratio≥6 to be combined. The signal-to-noise cutoff for peak selection was set at 20:1 for a minimum of two apexing masses. The baseline signal was drawn through the middle of the noise. To align peaks across chromatograms, the Statistical Compare software package in ChromaTOF® Version 4.72.0.0 (LECO Corp.) was utilized. Peaks were allowed to shift by a maximum of 18 seconds (9 modulation periods) in the first dimension and 0.15 seconds in the second dimension. The resulting aligned peaks were compared to the National Institute of Standards and Technology (NIST) 2011 Mass Spectral Library and tentative peak names were assigned if the spectral similarity score was ≥600 (60%). A secondary round of peak picking was performed on aligned chromatograms using a signal-to-noise threshold of 5 and a minimum spectral similarity≥60%. The resulting dataset was then manually filtered to remove poorly resolved peaks eluting before 400 seconds and known contaminants (siloxanes). Quantitative values for signal abundance were obtained by integrating peaks areas using the unique ion mass.

VOC identities were assigned according to the metabolomic reporting standards established previously [Sumner, L. W. et al., Metabolomics 3, 211-221 (2007)]. Compounds received an ID confidence level between 1 and 4, with 1 representing the highest confidence supported by two independent and orthogonal data sources [Sumner, L. W. et al., Metabolomics 3, 211-221 (2007)]. In brief, compounds with mass spectral match≥85%, to the NIST 2011 Mass Spectral Library, received an initial confidence level of 3. Compounds below this mass spectral threshold were assigned an ID level of 4 and labeled as unknown. The tentative VOC name and functional group is reported for all compounds with an ID level≤3. A C8-C20 alkane standard was analyzed and used to assign retention indices for all VOCs. An ID level of 2 was the highest classification in this study, verified with a ≥85% mass spectral match and a retention index that is consistent with the mid-polar Rxi-624Sil stationary phase using the mean of published RIs, according to an approach previously established [Bean, H. D. et al., Journal of breath research 10, 047102 (2016); Eshima, J. et al., Journal of Chromatography B 1121, 48-57 (2019)]. If mass spectral and polar second dimension chromatographic information supported the assigned of a functional group to a compound with an ID level of 4, then the compound was named by the functional group.

Statistical Analysis: To focus on the reproducible aspects of the biological volatilomes, compounds variably present (>20% missing observations) in the experimental and control volatilomes were removed, unless otherwise indicated. Integrated peaks were log₁₀ transformed in R, Version 4.0.3 (The R Foundation for Statistical Computing, Vienna, Austria) and missing values were imputed to 0 abundance. Pairwise, two-sided students' t-tests were applied comparing biological and control VOC abundances across the sampling duration. Abundance observations were assumed to be unpaired, with unequal variance. The resulting p-values were then adjusted according to the Benjamini-Hochberg procedure [Benjamini, Y. et al., Journal of the Royal statistical society: series B (Methodological) 57, 289-300 (1995)] using the “p.adjust” function in the base R “stats” library.

Sterilization and re-use Procedure: Following the conclusion of sampling and analysis in the Biodome, the TDT adapter was removed and submerged in a 100% ethanol bath for at least 1 hour and subsequently sonicated for 2 minutes. Removal of the live culture is dependent on the organism considered. For microbiological analysis, a freshly prepared 10% bleach solution was added directly through the outlet of the Biodome and left at room temperature for 15 minutes. For adherent cell culture, spent media was aspirated and 5 mL of 0.25% Trypsin/0.53 mM EDTA was added to the Biodome and incubated at 37° C. for 5-10 minutes to dissociate cells from the surface. Trypsinization was deemed a highly beneficial step to prevent accumulation of leftover cellular debris on the culture surface following autoclaving (later step). In both cases, the liquid culture was then emptied, and the Biodome was subject to two additional wash steps using bleach with significant lateral motion. Afterwards, the Biodome was flushed thoroughly with DI water and then autoclaved for 1 hour. Following sterilization in the autoclave, the biodome was then placed in an acid bath (3M HCl) overnight (≥10 hours), although 3 hours was found to be sufficient. Following the acid bath, the Biodome was flushed with DI water and transferred to a base bath (approximately 150 mM NaOH) for at least 3 hours. Finally, to reduce noise from the culture vessel, the Biodome was flushed again with DI water, placed in a glass beaker, and heat treated for >12 hours at 100° C. In some embodiments, treating the Biodome in the base bath second, to limit the detection of acid-related contaminants.

The following publications are incorporated by reference:
U.S. Publication No. US20220331803A1, published on Oct. 20, 2022

The disclosures of each and every patent, patent application, and publication cited herein are hereby each incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A tray device, comprising a frame comprising a top surface, a bottom surface, and a thickness,
wherein the top surface of the frame comprises:
a first recessed region having a sidewall around at least a portion of a perimeter, and a ledge extending laterally from the bottom of the sidewall of the first recessed region, wherein the sidewall defines at least a portion of a first shape;
a second recessed region having a sidewall around at least a portion of a perimeter, and a ledge extending laterally from the bottom of the sidewall of the second recessed region, wherein the sidewall defines at least a portion of a second shape; and
wherein the depth of the first recessed region is deeper into the thickness of the tray than the depth of the second recessed region.

2. The device of claim 1, further comprising a third recessed region within the top surface of the frame,
wherein the third recessed region has a sidewall around at least a portion of a perimeter and a ledge extending laterally from the bottom of the sidewall, and
wherein the sidewall defines at least a portion of a third shape.

3. The device of claim 2, wherein the depth of the second recessed region is deeper into the thickness of the tray than the depth of the third recessed region.

4. The device of claim 1, wherein the first shape at least partially resembles the shape of a biodome device.

5. The device of claim 1, wherein the second shape at least partially resembles the shape of a culture flask.

6. The device of claim 2, wherein the third shape at least partially resembles the shape of a culture flask.

7. The device of claim 1, wherein the sidewall of the first recessed region has a height ranging from about 0.1 mm to about 10 mm.

8. The device of claim 1, wherein the sidewall of the second recessed region has a height ranging from about 0.1 mm to about 10 mm.

9. The device of claim 2, wherein the sidewall of the third recessed region has a height ranging from about 0.1 mm to about 10 mm.

10. The device of claim 1, wherein the first recessed region at least partially overlaps with the second recessed region.

11. The device of claim 1, wherein the perimeter of the first recessed region is less than the perimeter of the second recessed region.

12. The device of claim 2, wherein the first recessed region and the second recessed region are encompassed entirely within the perimeter of the third recessed region.

13. The device of claim 2, further comprising an opening that passes through the first, second, and third recessed regions.

14. The device of claim 2, wherein the frame comprises an opaque and rigid material.

15. The device of claim 2, further comprising one or more handles extending up vertically from the top surface of the frame.

16. The device of claim 1, wherein it is used for imaging a biological sample within a biodome.

17. The device of claim 16, wherein the biological sample consists of a bacterial cell.

18. The device of claim 16, wherein the biological sample consists of a mammalian cell.

19. A method of using a device of claim 1 for fluorescent imaging of a biological sample to measure volatile organic compounds (VOCs).

20. The method of claim 19, further comprising a dynamic headspace sampling methodology and computational modeling.


SEQUENCE LISTING (12 sequences)

SEQ ID NO: 1 - RNA, 19 nt, synthetic construct
  source 1..19: mol_type = other RNA
ggcagaagctgatgagtag

SEQ ID NO: 2 - RNA, 19 nt, synthetic construct
  source 1..19: mol_type = other RNA
tggcatacctgaccagtga

SEQ ID NO: 3 - RNA, 19 nt, synthetic construct
  source 1..19: mol_type = other RNA
ggaaagtggactcggcagt

SEQ ID NO: 4 - RNA, 19 nt, synthetic construct
  source 1..19: mol_type = other RNA
caagagactcatacgcttt

SEQ ID NO: 5 - RNA, 19 nt, synthetic construct
  source 1..19: mol_type = other RNA
tagcgactaaacacatcaa

SEQ ID NO: 6 - RNA, 19 nt, synthetic construct
  source 1..19: mol_type = other RNA
taaggctatgaagagatac

SEQ ID NO: 7 - RNA, 19 nt, synthetic construct
  source 1..19: mol_type = other RNA
atgtattggcctgtattag

SEQ ID NO: 8 - RNA, 19 nt, synthetic construct
  source 1..19: mol_type = other RNA
atgaacgtgaattgctcaa

SEQ ID NO: 9 - DNA, 20 bp, synthetic construct
  source 1..20: mol_type = other DNA
tggtttatctgcccgtatgc

SEQ ID NO: 10 - DNA, 18 bp, synthetic construct
  source 1..18: mol_type = other DNA
tgctccaagtaccatggc

SEQ ID NO: 11 - DNA, 19 bp, synthetic construct
  source 1..19: mol_type = other DNA
acatcgctcagacaccatg

SEQ ID NO: 12 - DNA, 22 bp, synthetic construct
  source 1..22: mol_type = other DNA
tgtagttgaggtcaatgaaggg